(19) United States
(12) Patent Application Publication
(10) Pub. No.: US 2007/0110759 A1
(43) Pub. Date: May 17, 2007

(54) Methods

(75) Inventors: Quentin James Sattentau, Oxford (GB); Peter John Openshaw, London (GB); Amin Eisazadeh Moghaddam, Oxford (GB); Wieslawa Olszewska, London (GB)

Correspondence Address: NIKOLAI & MERSEREAU, P.A., 900 SECOND AVENUE SOUTH, SUITE 820, MINNEAPOLIS, MN, 55402, US

(73) Assignee: Imperial Innovations Limited, London (GB)

(21) Appl. No.: 11/434,320

(22) Filed: May 11, 2006

Related U.S. Application Data
(63) Continuation of application No. PCT/GB04/04928, filed on Nov. 15, 2004.

(30) Foreign Application Priority Data
Nov. 13, 2003 (GB) 0326439.7

(51) Int. Cl. A61K 39/00; C08B 37/00; C07K 14/82

(52) U.S. Cl. 424/184.1; 424/277.1; 530/350; 536/53

(57) ABSTRACT

The presence of aldehydic groups on proteins and lipoproteins is associated with various pathological conditions such as atherosclerosis, diabetes and alcoholic liver disease. Respiratory syncytial virus (RSV) is a major cause of severe respiratory disease in infants and the elderly. RSV vaccine research has been impeded because a formalin-inactivated vaccine used in the 1960s predisposed infants to enhanced disease following subsequent natural infection. The molecular basis for the vaccine-induced hypersensitivity has not, however, been elucidated. We show here that addition of reactive carbonyl groups to ovalbumin (OVA) by treatment with glycolaldehyde or formaldehyde increases the protein's immunogenicity in mice, and biases the immune response towards a Th2-type response. The increased immunogenicity and the Th2-type response can both be abrogated by reductive elimination of the reactive carbonyl groups. We demonstrate that RSV inactivated by formaldehyde (FI-RSV), following a protocol used previously to prepare the vaccine, contains reactive carbonyl groups. Using a well-established model of FI-RSV vaccine-induced pathology, immunisation of mice with FI-RSV and subsequent challenge of the mice with live RSV induced Th2-type responses, lung eosinophilia and weight loss that were abrogated by reductive elimination of the reactive carbonyl groups. We thus propose that the addition of reactive carbonyl groups to RSV during inactivation is the major mechanism that drives the Th2-immune response and associated pathology. Moreover, we suggest that the addition of reactive carbonyl groups to other antigens, including vaccines, may be responsible for other hypersensitive and allergic reactions described in the literature.

The present invention relates to a method of modifying an antigen to modify the Th2-type bias of the Th1/Th2-type immune response of an animal, for example a human, exposed to the antigen.

The adaptive immune response to antigenic stimulation can be divided into two broad types, termed T-helper type-1 (Th1) and T helper type-2 (Th2). These responses cover a large spectrum of immune reactivity to antigens, defined principally by the types of cytokines secreted by T cells during the response. In general terms, a response biased towards a Th1 type is typified by secretion of interferon gamma and interleukin-12 (IFN-γ and IL-12 respectively) cytokines. A Th1 response will tend to accompany strong CD8+ killer T cell responses and so is important for immunity against intracellular pathogens such as viruses, intracellular bacteria eg. Tuberculosis, Mycobacteria and Salmonella spp., intracellular parasites and yeasts. If uncontrolled, Th1 cells can mediate immunopathology and have also been implicated in autoimmune diseases such as type-1 diabetes, multiple sclerosis, rheumatoid arthritis, experimental autoimmune encephalitis, and others (reviewed in O'Garra and Arai, (2000) Trends Cell Bio 10, 542-550).

A Th2 bias in the immune response is typified by a different balance of cytokine production from a Th1 bias. Thus Th2 cells produce a profile of cytokines including, for example, IL-4, 5, 9 and 13 that together instruct B cells to proliferate and differentiate into antibody—secreting plasma cells, and potentiate the function of several cell types in antiparasite responses. Some Th2 cytokines (for example, IL-4) antagonise the production of Th1-type cytokines, whereas some Th1-type cytokines (for example IFN-γ) antagonise the production of Th2-type cytokines. In contrast to Th1 cells protecting against intracellular pathogens, Th2 cells play an important role in providing protection against certain extracellular pathogens including intestinal helminths. However, these cells can also mediate allergic and atopic manifestations, which is in keeping with findings that Th2-derived cytokines can induce airway hyperreactivity (for example, asthma) and the production of IgE (reviewed in Dong and Flavell, (2000) Arthritis Res 2, 179-188).

Both Th1- and Th2-specific cytokines can promote growth or differentiation of their own respective T-cell subset, but additionally can inhibit the development of the opposing subset. Th1 cells produce IFNγ, which will inhibit the proliferation of Th2 cells, whereas Th2 cells produce interleukin-4 (IL-4), which inhibits the production of IFNγ by Th1 cells (de Waals Malefyt, (1997), Semin Oncology 3, suppl 9, S9-94-S9-98). This might explain why Th1 and Th2 responses are often mutually exclusive, although many responses are a balance between the two types.

Hypersensitivity is one of a class of immune system responses that may be defined as exaggerated or unwanted immune responses to exogenous antigens. These are harmful immune responses that produce tissue injury and may cause serious disease, including allergy. Hypersensitivity responses are classified as type-I to type IV depending on the immune mechanisms.

Allergy can manifest itself in a wide range of symptoms affecting any organ in the body. Allergy to ingested substances commonly affects particularly the gastrointestinal tract, the skin, the lung, the nose and the central nervous system. Allergic reactions to ingested substances affecting these organs can manifest themselves as abdominal pain, abdominal bloating, disturbance of bowel function, vomiting, rashes, skin irritation, wheezing and shortness of breath, nasal running and nasal blockage, headache and behavioural changes. In addition, in severe allergic reactions the cardiovascular and respiratory systems can be compromised with anaphylactic shock and in some cases death.

It is also recognised that in certain chronic diseases, allergy to ingested substances is the probable cause of the disease in a proportion of patients. These diseases include susceptibility to anaphylactic shock, atopic dermatitis, chronic urticaria, asthma, allergic rhinitis, irritable bowel syndrome, migraine and hyperactivity in children. It is also possible that food allergy may be a factor in certain patients with inflammatory bowel disease (ulcerative colitis and Crohn's disease).

Allergy to inhaled substances can manifest itself as rhinitis, asthma or hayfever. The respiratory tract and/or eyes may be affected. For example, asthma can be provoked by inhalation of allergen in the clinical laboratory under controlled conditions. The response is characterised by an early asthmatic reaction (EAR) a manifestation of a type I hypersensitivity response followed by a delayed-in-time late asthmatic reaction (LAR) a typical type IV hypersensitivity response (See Allergy and Allergic Diseases (1997), A. B. Kay (Ed.), Blackwell Science, pp 1113 to 1130). The EAR occurs within minutes of exposure to allergen, is maximal between 10 and 15 min and usually returns to near baseline by 1 hour. It is generally accepted that the EAR is dependent on the IgE-mediated release of mast cell-derived mediators such as histamine and leukotrienes. In contrast the LAR reaches a maximum at 6-9 hours and is believed to represent, at least in part, the inflammatory component of the asthmatic response and in this sense has served as a useful model of chronic asthma.

The late asthmatic response is typical of responses to allergic stimuli collectively known as late phase responses (LPR). LPR is seen particularly in the skin and the nose following intracutaneous or intranasal administration of allergens.

Allergy by skin contact may manifest itself as eczema or atopic dermatitis. Atopic dermatitis is an inflammatory skin disorder, affecting up to 10% of the paediatric population. It is characterised by itching, a chronic relapsing course and typical distribution around the body. There is usually a family history of allergy and the condition starts in early infancy. Typical treatment regimes are to use simple emollients or topical corticosteroids. Long-term use of topical corticosteroids may have undesirable side effects, particularly in children. Contact allergens include latex, detergents or other ingredients of washing powders, animal dander and house dust mites.

Allergic reactions occur when an individual who has produced IgE antibody in response to an allergen subsequently encounters the same allergen. Allergens are antigens that elicit hypersensitivity or allergic reactions. The allergen triggers the activation of IgE-binding mast cells in the exposed tissue, leading to a series of responses that are characteristic of allergy. There are circumstances in which IgE is involved in protective immunity, especially in response to parasitic worms, which are prevalent in less developed countries. In the industrialised countries, however, IgE responses to innocuous antigens predominate and allergy is an important cause of disease. Because of the medical importance of allergy in industrialised societies, much more is known about the pathophysiology of IgE-mediated responses than about the normal physiological role of IgE.

IgE production is driven by the Th2-class of Th cells in type-I hypersensitivity reactions.

Since IgE production is driven by Th2 cells and Th2 cell-derived cytokines can induce airway hyperreactivity (for example, asthma) it is clear that Th2 cells play a central role in mediating the immune response to an allergen.

The therapy of allergic disease is currently chiefly symptomatic, with agents such as anti-histamines, β₂ agonists and glucocorticosteroids most commonly used. However, this has no impact on the underlying abnormal immune response or its cause.

We show that the presence of reactive carbonyl groups such as aldehydes on an antigen can lead to a Th2-type biased immune response and so induces a hypersensitivity response. We show that, for example, decreasing the number of reactive carbonyl groups decreases the Th2 bias. We provide, for example, a method for a simple, one step reductive elimination of reactive carbonyl groups from an antigen. This method is of particular use in reducing the allergenicity of an agent to be administered to an animal (such as a human), for example a vaccine or therapeutic agent.

We also show that, for example, increasing the number of reactive carbonyl groups increases the Th2 bias. We provide, for example, a method for a simple, one step addition of reactive carbonyl groups to an antigen. This method is of particular use in disease states where an unwanted Th1 response is present, or in vaccines where a Th2-type response is advantageous, such as anti-parasite vaccines.

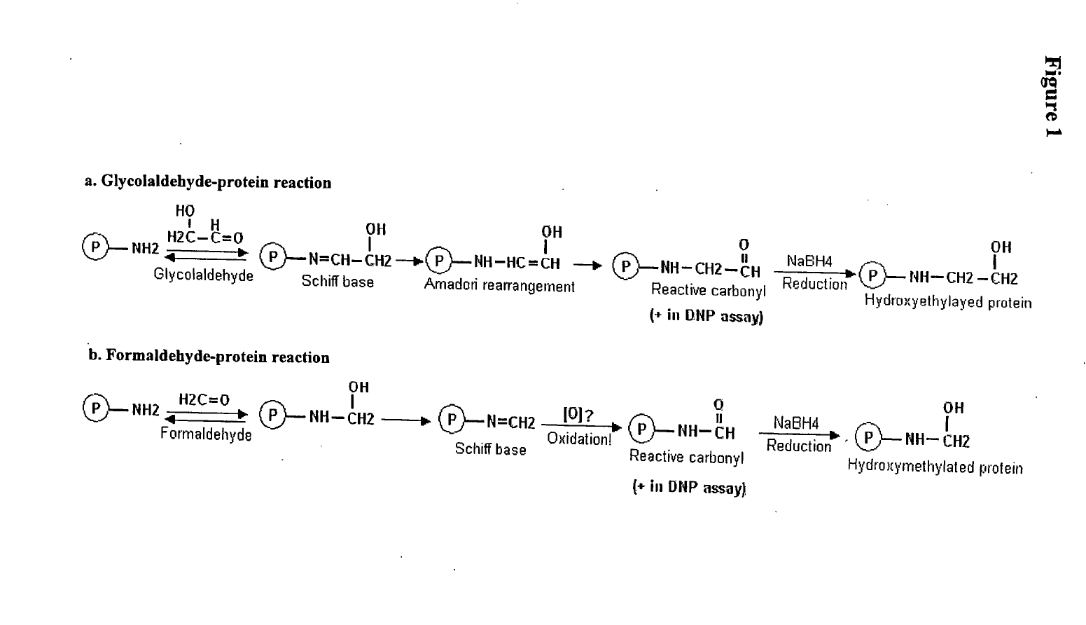

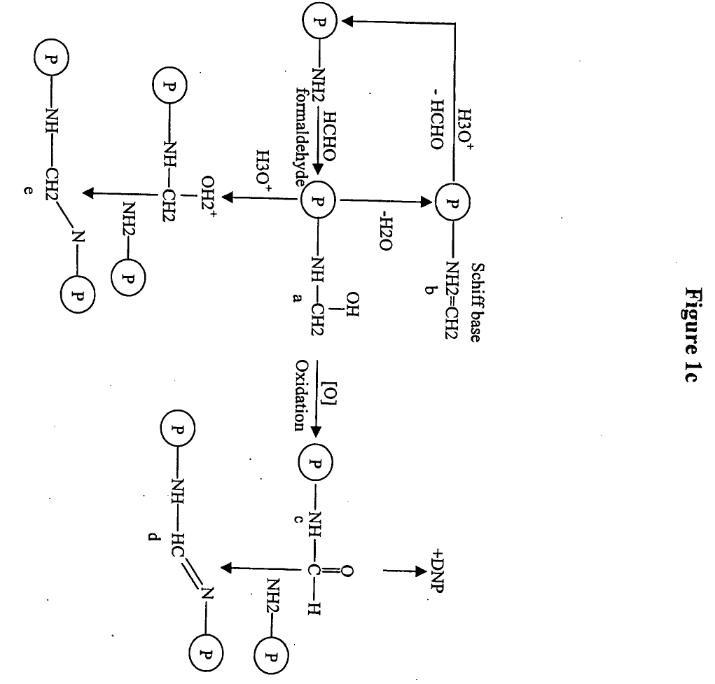

Reactive carbonyl groups are carbon atoms double-bonded to an oxygen atom and single-bonded to two other groups or atoms. Examples of such groups include ketones and aldehydes. When one of the groups is a hydrogen, as in aldehydes (R—HC═O) the polarity of C═O increases which makes the carbonyl group highly reactive (FIG. 1).

By ‘reactive carbonyl group’ we include all reactive species described above as well as precursor chemical forms to reactive carbonyls and intermediate chemical forms during aldehyde reactions with proteins, such as Schiff bases which could be reduced by NaBH3CN or NaBH₄. The presence of reactive carbonyls can be detected using a well-established assay based on the reactivity of 2,4-dinitrophenyl-hydrazine (DNPH) with the carbonyl group of the aldehyde.

The immune-potentiating (adjuvant) properties of aldehyde groups and aldehyde-antigen adduction have been previously described. However, it has not been reported that the presence of aldehyde groups on an antigen may be responsible for the antigen acting to induce a hypersensitive response, ie a Th2-type biased immune response, in an animal exposed to the antigen.

Reactive carbonyl groups may be present naturally in an antigen and have been implicated in evoking an immune response. For example, QS-21, a purified saponin immunogenic adjuvant, contains an aldehyde on the triterpene. Soltysik et al (1995) Vaccine 13, 1403-1410 demonstrate that QS-21 derivatives modified at the aldehyde group do not show adjuvant activity for antibody production or for the induction of cytotoxic T-lymphocytes, suggesting that this functional group may be involved in the adjuvant mechanism.

Also known is that the addition of aldehyde groups to antigens or adjuvants can increase their immunogenicity. For example, Allison and Fearon (2000) Eur J Immunol 30, 2881-2887 describe how the introduction of aldehydes into poorly-immunogenic antigens by glycoaldehyde treatment enhances by several orders of magnitude their immunogenicity in terms of antibody production in mice. Furthermore, WO 99/53946 report that the introduction of aldehydes into antigens results in enhanced antibody responses indicative of both a Th1 and Th2 response. Apostolopoulos et al (1995) Proc Natl Acad Sci USA 92, 10128-10132 report that creating aldehydes on an antigen by coupling the antigen to peroxide-oxidised mannan enhances its ability to elicit cytotoxic T cells and a Th1 response. However, in this particular example the mannan is likely to bias a Th1 immune response due to its recognition by receptors of the innate immune response. This may, therefore explain the Th1 bias in the presence of aldehydes.

As well as directly modifying the antigen or adjuvant, there have been several reports that supplying aldehyde-generating drugs with an antigen leads to an increase in immune response (Rhodes et al (1995) Nature 377, 71-75; Zheng et al (1992) Science 256, 1560-1563).

Finally, Willis et al (2002) Alcohol Clin Exp Res 26, 94-106 and Willis et al (2003) Int Immunopharmacol 3, 1381-1399 report that the presence of malondialdehyde-acetaldehyde adducts (MAA) induces antibody and T-cell proliferative responses via scavenger receptors in-vivo.

Reactive carbonyl groups (including aldehyde groups) may be generated on proteins by aldehyde treatment. However, it is important to note that the treatment of antigens with aldehydes results in various antigen-aldehyde adducts, some of which have reactive carbonyls, others of which are non-reactive, non-aldehydic end-products. Adduction of antigens with aldehydes results in various structures, some of which are stable and some of which are unstable. The variety and proportion of these adducts would, apart from the type of aldehyde used, depend on physicochemical indices such as, for example, the type of antigen, pH, temperature and duration of reaction.

For example, Acharya and Manning (Proc. Natl. Acad. Sci. 80, 3590-3594; 1983) have studied a number of glycolaldehyde-protein adducts, of which only the “2-oxoethylated protein” has an aldehyde group.

Also, during the Maillard reaction (a reaction between reducing sugars and amino structures in amino acids or proteins), proteins are modified which results in the production of various aldehyde-protein adducts, not all of which bear aldehyde groups. Carboxymethyl-lysine (CML), for example, is a product of a protein-aldehyde reaction and does not have aldehyde groups (Glomb and Monnier, (1995) J Biol Chem 270, 10017-26).

Thus, reactive carbonyl groups are just a part of the adducted structure on an antigen. In this regard very few immunopathological studies have specifically focused on the importance of reactive carbonyl groups rather than the whole new added structure on proteins (of which the aldehyde group may be just a moiety).

Furthermore, the prior art does not suggest modulating the number of reactive carbonyl groups present in an antigen to vary the ability of the antigen to induce hypersensitivity. Moreover Willis et al supra discuss haptenated proteins (aldehydes being the haptens that upon adduction by proteins become immunogenic), while the present invention relates to the immunomodulatory effects of adding or removing reactive carbonyl groups on the type of immune response to the antigen.

A first aspect of the invention provides a method of modifying an antigen to modify the Th2-type bias of the Th1/Th2-type immune response of an animal exposed to the antigen, the method comprising:
(i) decreasing the number of reactive carbonyl groups present in the antigen so as to decrease the Th2-type bias; or,
(ii) increasing the number of reactive carbonyl groups present in the antigen so as to increase the Th2-type bias.

As will be outlined below, the method of the first aspect of the invention may be used to decrease the number of reactive carbonyl groups present in the antigen, ie the decreasing option. This may have particular utility in reducing the hypersensitivity of a patient to an antigen, including a vaccine. The first aspect of the invention may also be used to increase the number of reactive carbonyl groups present in the antigen, ie the increasing option. This may have particular utility in increasing the Th2-inducing immunogenicity of the antigen.

Examples of how the method of the first aspect of the invention may be used to modify an antigen to modify the Th2-type bias of the Th1/Th2-type immune response are presented in the accompanying examples.

By ‘decreasing the number of reactive carbonyl groups present in the antigen’ we mean that the number of reactive carbonyl groups is reduced by 10%, 25%, 50%, 60%, 70%, 80%, 90%, 95%, 99%, 99.5% or more, for example 100%. Methods of measuring the number of reactive carbonyl groups present in the antigen are set out below and in the accompanying examples.

By ‘decrease the Th2-type bias’ we mean that an animal exposed to an antigen having been modified according to the method of the first aspect of the invention (decreasing option) will develop an immunogenic response which is more in character with that induced by Th1 cells or of a balanced Th1/Th2 response than by Th2 cells, when compared to the immune response of an animal exposed to the antigen which has not been so modified. The modified antigen may exhibit reduced overall immunogenicity.

It is preferred that one or more indicators of the Th1/Th2-cell type ratio discussed below (for example, relative levels of IgG1 to IgG2a antibodies in mice) indicate that there is a change in the Th1/Th2 cell-type ratio evoked by the modified antigen. Such a change may be in the order of a 1.2×, 1.5×, 1.8×, 2×, 3×, 5×, 10×, 20×, 30×, 50×, 70× or 100× increase in the Th1/Th2 cell-type ratio in favour of Th1 cells relative to the Th1/Th2 cell-type ratio evoked with the untreated antigen (ie retaining reactive carbonyl group(s)). This does not exclude the possibility that there may still be a Th2-bias in the Th1/Th2-cell type ratio.

By ‘increasing the number of reactive carbonyl groups present in the antigen’ we mean that the number of moles of reactive carbonyl groups present on a mole antigen are increased from 0 to 0.1, 0.5, 1, 2, 3, 4, 5, 6, 7, 8, 9 or 10 moles or more. Methods of measuring the number of reactive carbonyl groups present in the antigen are set out below and in the accompanying examples.

By ‘increase the Th2-type bias’ we mean that an animal exposed to an antigen having been modified according to the method of the first aspect of the invention (increasing option) will develop an immunogenic response which is more in character with that induced by Th2 cells than by Th1 cells, when compared to the immune response of an animal exposed to the antigen which has not been so modified. The antigen may exhibit increased overall immunogenicity.

It is preferred that one or more indicators of the Th1/Th2-cell type ratio discussed below (for example, relative levels of IgG1 to IgG2a antibodies in mice) indicate that there is a change in the Th1/Th2 cell-type ratio evoked by the modified antigen. Such a change may be in the order of a 1.2×, 1.5×, 1.8×, 2×, 3×, 5×, 10×, 20×, 30×, 50×, 70× or 100× increase in the Th1/Th2 cell-type ratio in favour of Th2 cells relative to the Th1/Th2 cell-type ratio evoked with the untreated antigen (ie without the added reactive carbonyl groups). This does not exclude the possibility that there may still be a Th1-bias in the Th1/Th2-cell type ratio.

A modified Th2-type response may be shown when the ratio of IgG2a antibody concentrations to IgG1 antibody concentrations for a chosen antigen increases or decreases in mice. Increased IgG2a/IgG1 concentrations correlate with reduced Th2 profiles in mice and vice versa (Mosmann, T. R., and Coffman, R. L. (1989) Annu Rev Immunol 7, 145-173).

Proliferation of lymph node cells, IFNγ production, IL5 and/or IgE levels, or levels of other cytokines, should also be used in assessing the Th1/Th2-cell type ratio, particularly in humans. For example, to examine antigen-specific T cell responses, an ex vivo assay that measures the proliferation of lymph node cells of human or animal origin (Alkan (1978) Eur J Immunol 8, 112-8) may be used. This assay is chiefly used for Th1 responses as it is dependent on T cell proliferation and L-2 production. Lymph node cell cultures may also be used to measure Th1/Th2-cell type cytokine profiles, by analysis of the cell supernatant, intracellular FACS staining or by the use of Elisa-spot technology (Elispot). A high relative level of IFNγ and/or TNF and/or IL-12 production and a low relative level of IL-4 and/or IL-5 and/or IL-13 production is indicative of a Th1 cell-type response, whilst a high relative level of IL-4 and/or IL5 and/or IL-13 and a low relative level of IFN-γ and/or TNF and/or IL-12 production is indicative of a Th2 cell-type response.

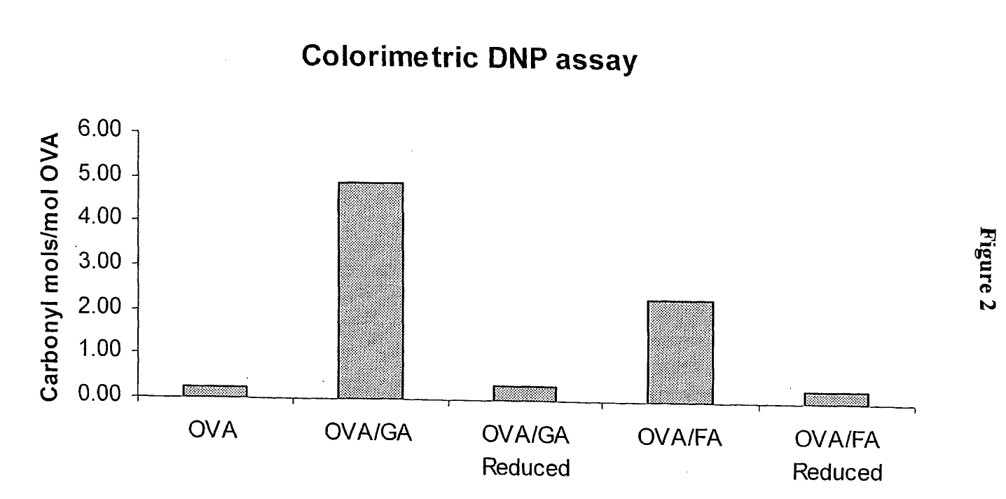

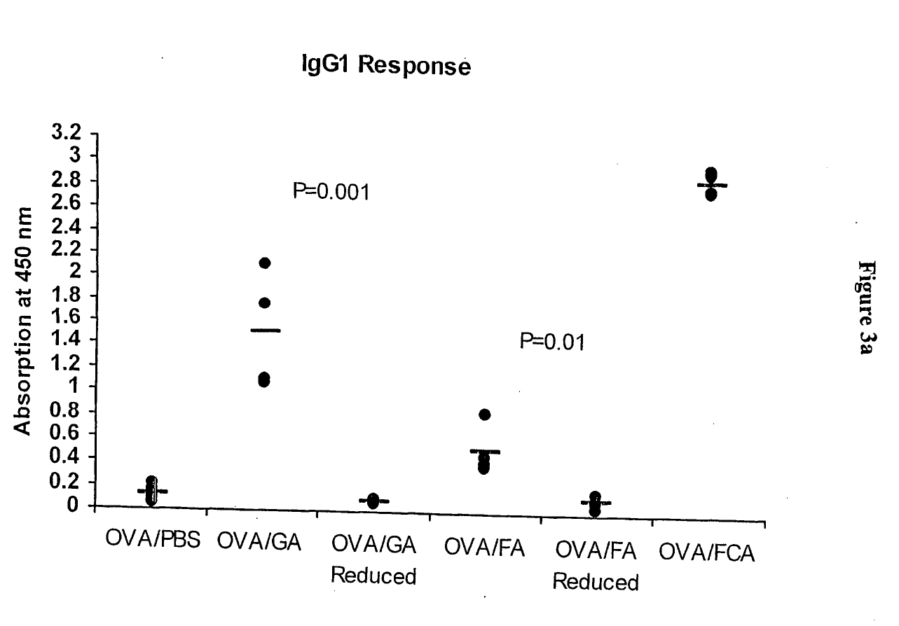

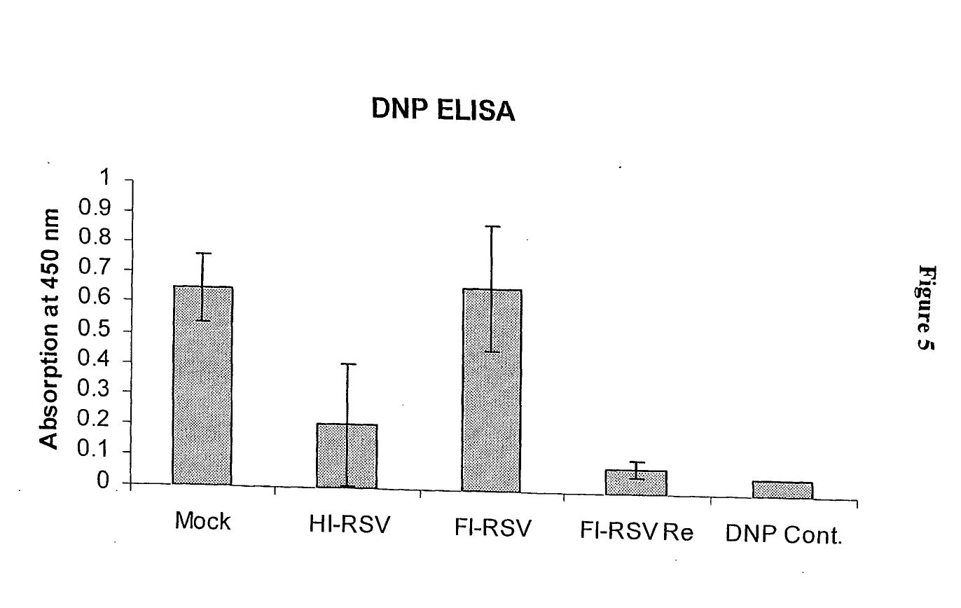

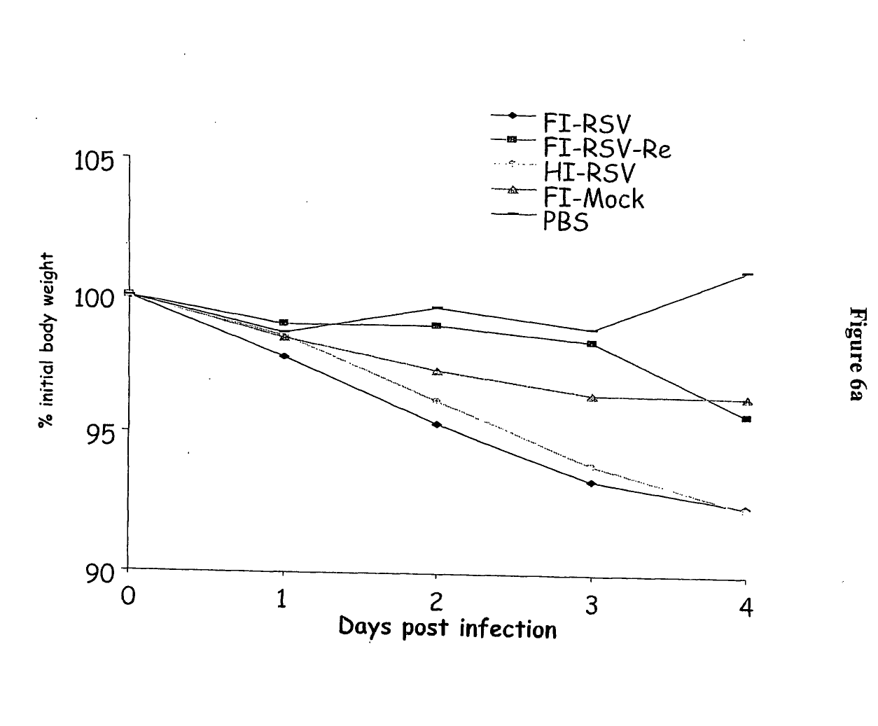

Methods of measuring the number of reactive carbonyl groups present in the antigen include those shown in the accompanying examples. Suitable methods include:
1. Immunometric measurements based on an ELISA method published by Buss et al (1997) Free Radical Biology & Medicine 23, 361-66. The following modified method may be used:
   10 μl sample (2-10 μg protein)+40 μl DNP 10 mM in 2M HCl, Mix well, incubate for 45 min with shaking
   5 μl of mix (1-5 μg protein)+95 μl of coating buffer (NaHCO3, pH=8.5)
   Coat ELISA plate with 100 μl/well of the above solution and incubate at 4° C. over night (or 90 minutes at 37° C.)
   Wash ×3 with PBS
   Add 200 μl blocking buffer (BSA1%/PBS), incubate for 30 minutes at room temperature
   Wash as above
   Add 100 μl anti-DNP biotinylated Ab (1/1000), incubate at room temperature for one hour
   Wash as above
   Add to 100 μl streptavidine-HRP (1/1000), incubate at room temperature for one hour
   Wash as above
   Add 100 μl substrate (TMB ultra)
   Stop reaction at the appropriate time with 100 μl H₂SO₄
   Read at 450 nm
2. Western blotting as described by Robinson et al (1999) Analytical Biochemistry 266, 48-57. This method was used in the accompanying example to generate the data shown in FIG. 5.
3. Spectrophotometric or colourimetric DNPH assay as used in the accompanying example to generate the data shown in FIG. 2.
4. Spectrophotometric DNPH assay coupled to protein fractionation by HPLC.
5. Mass spectrometry may also be used to measure any change in the number of reactive carbonyl groups present in the antigen.

Any suitable method may be used to decrease the number of reactive carbonyl groups present in the antigen. Suitable methods include:
1. Reaction of the antigen with potent reducing agents such as hydrides, including NaBH₄, NaCNBH₃, dimethylamine borane or piridine borane. A detailed protocol for this method is shown in the accompanying examples and presented below:
The reactive carbonyl groups can be reduced either during incubation of the antigen with the aldehyde or subsequent to reactive carbonyl addition, by incubation of the antigen with 0.1 mM NaBH₄. for 1-3 hours at room temperature or at 37° C. The samples are then desalted following manufacturers instruction using Microcon® 10 kDa cutoff microspin filters.
2. Hydrogenation of the antigen in the presence of an appropriate catalyst. Eg. CH═O+H₂ yields CH₂—OH, which is a non-reactive hyroxymethyl end product. (see Organic chemistry II-reduction of aldehydes/ketones). A detailed protocol for this method may be found in the accompanying examples.
3. Other methods for eliminating or reducing numbers of reactive carbonyls are to use aldehyde-sequestering or scavenging drugs or agents such as glutathione, (see Dickinson et al, Glutathione in defense and signalling: lessons from a small thiol. Ann. NY. Acad. Sci. 973: 488-504 (2002) aminoguanidine and pyridoxamine (see: Burcham et al, Aldehyde-sequestering drugs: tools for studying protein damage by lipid peroxidation products—Toxicology: 181-182, 229-236 (2002)). Also, anti-oxidants such as camosine (see: Hipkiss, A R, Carnosine, a protective, anti-ageing peptide?, Int. J. Biochem. Cell Biol. 30, 863-868 (1998)), melatonin and N-acetylcysteine (see Sener et al, Melatonin and N-acetylcysteine have beneficial effects during hepatic ischemia and reperfusion, Life Sciences 72: 2707-2718 (2003), pyruvate (see Varma et al, Oxidative damage to mouse lens in culture. Protective effect of pyruvate. Biochem. Biophysica Acta 1621: 246-252 (2003) and copper, zinc, tellurium and selenium metal ions (see Klotz et al Emerging functional endpoints of trace element status, J. Nutr. 133: 1448S-1451S (2003)) may be used to prevent or repair oxidative damage leading to generation of reactive carbonyls.

Any suitable method may be used to increase the number of reactive carbonyl groups present in the antigen. Methods include those set out in the accompanying examples, such as treatment with aldehydes such as glycolaldehyde, acetaldehyde, malonaldehyde and formaldehyde. Further examples of suitable methods include oxidation of carbohydrates on glycoproteins using, for example, NaIO₄ and the reaction of proteins with reducing sugars via the Maillard reaction; also, UV light, ozone, nitrogen oxides, radiation, neutrophil activity (via the myeloperoxidase pathway), metal catalysed oxidative pathway, hyperchlorous acid and peroxynitrite oxidation, and enzymatic modification of amino acids. For examples of these methods see Adams et al “Reactive Carbonyl formation by oxidative and non-oxidative pathways” Frontiers in Bioscience 6: 17-24 (2001).

The animal exposed to an antigen having been modified according to the method of the first aspect of the invention may be a mammal, for example a human, cow, pig, goat, horse, sheep, dog, cat, mouse, rat, rabbit, guinea pig or a wild species, such as foxes or badgers, for which vaccination may be used to protect domestic species, or the like. The animal may be very young, maturing, mature or senescent. Where the animal is a human, the human may be an adult or a child and may be either male or female. Alternatively, the animal may be a bird, for example a chicken, turkey or other such poultry. Preferably the animal is a human.

An antigen is a substance which can induce an immune response and may be any naturally occurring, recombinant or synthetic product. The term antigen also includes complexes of protein carriers and non-protein molecules such as steroids, carbohydrates or nucleic acids, wherein the complex is used as an immunogen for the production of an immune response to the non-protein molecule.

An embodiment of the first aspect of the invention is wherein the antigen is or comprises a protein, glycoprotein, lipoprotein, polysaccharide, or a nucleic acid.

The antigen may be derived from a range of natural or synthetic sources. Synthetic sources may include latex and protein detergent additives.

Alternatively, the antigen or part thereof may be derived from a mammalian cell, plant cell, bacteria, virus, fungus or parasite. The antigen may be or comprise a tumour antigen or an autoantigen, ie the antigen may be one that is present in the intended recipient. The antigen may be derived from a live or killed organism.

As discussed above, the first aspect of the invention provides a method of decreasing the number of reactive carbonyl groups present in an antigen, ie the removing step, to decrease the Th2 bias of the immune response to the antigen. Examples of antigens for which it may be useful to decrease the Th2 bias of the immune response include antigens for which autoreactivity is seen, such as in liver or pancreas pathology induced by alcohol consumption that leads to reactive carbonyl adduction of self antigens or autoreactive lung pathology induced by cigarette smoke that contains aldehydes including formaldehyde. The modified antigen may be used for desensitisation treatment, which is discussed further below.

In addition, sequestering/scavenging agents and anti-oxidants described above in relation to methods of removing reactive carbonyl groups from antigens may used systemically or locally to reduce the number of free aldehydes or to reduce the number of reactive carbonyl adducts on antigens in vivo.

Alternatively, the first aspect of the invention provides a method of increasing the number of reactive carbonyl groups present in an antigen, ie the adding step, to increase the Th2 bias of the immune response to the antigen.

In some cases, for example, rheumatoid arthritis, an increased Th1 immune response leads to pathology. Hence an application of the method of the first aspect of the invention may be the use of an agent, for example an aldehyde, that increases the number of reactive carbonyl groups in an antigen so as to bias the local immune response towards a more benign Th2-type response. For example, weak aldehydes may be injected into a site of arthritic inflammation to induce a Th2 bias. Similar applications may be relevant to other autoimmune Th1 biased pathologies.

An embodiment of the first aspect of the invention is where the antigen is a vaccine or vaccine component.

The method of the first aspect of the invention may be used to decrease the number of reactive carbonyl groups present in the vaccine or vaccine component, ie the decreasing option. Examples of possible vaccines or vaccine components for which this may be desirable include those which are formaldehyde-treated, as set out below.

Alternatively, the method of the first aspect of the invention may be used to increase the number of reactive carbonyl groups present in the vaccine or vaccine component, ie the increasing option. Some vaccines may benefit from an increased Th2 response, for example, vaccines to parasitic helminths such as schistosoma and filaria. Here a potent Th2-type immune response including IgE production may be required for protection against the pathogen (see MacDonald et al, Immunology of parasitic helminth infections, Infection and Immunity 70: 427-433 (2002) and Meeusen and Piedrafita, Exploiting natural immunity to helminth parasites for the development of veterinary vaccines, Int. J. Parasitol. 33: 1285-1290 (2003)). Therefore, incorporation of reactive carbonyls into vaccine antigens would increase immunogenicity and bias the immune response towards a Th2-type response. If effective, such vaccines would have importance for human and animal health.

Formaldehyde treatment has been a standard means of inactivating, stabilising and preserving vaccines for pathogenic agents, for example viruses and bacteria. However, some vaccines can evoke a hypersensitivity response in some patients. As can be seen from the accompanying examples, we have shown that this may be due to the presence of reactive carbonyl groups on the vaccine which are a result of the formaldehyde treatment. Therefore, it may be possible to reduce any hypersensitivity response a patient may display towards such a vaccine by first by modifying formaldehyde treated vaccines to remove the reactive carbonyl groups.

Accordingly, a further embodiment of this aspect of the invention is where the vaccine or vaccine component has been formaldehyde-treated prior to being modified by the method of the first aspect of the invention. The method of the first aspect of the invention may be used to decrease the number of reactive carbonyl groups present in an antigen, ie the decreasing option. Examples of vaccines which may be modified according to the decreasing option of the first aspect of the invention include those vaccines to Respiratory Syncytial Virus, Measles, Influenza, human metapneumavirus, Hantavax (a commercial vaccine against Hantaviruse, causative agent of haemorrhagic fever with renal syndrome (HFRS)), WEE, EEE, VEE (Western, Eastern and Venezuelan Equine Encephalitis), encephalitis viruses, anthrax, mumps, pertussis, viral hepatitis, meningitis, poliomyelitis, tuberculosis, rubella, tetanus, diptheria, coronavirus infections or other local or systemic infection of animals or man.

Antigens present in certain foodstuffs may be allergenic, for example, fish, shellfish, crab, lobster, peanuts, nuts, wheat gluten, eggs and milk. In particular, as set out above, nut allergies can induce a severe immune reaction in some individuals and may lead to anaphylactic shock and in some cases death.

The preparation of some foodstuffs may generate reactive carbonyl groups on proteins. For example, reactive carbonyl groups can be added to foodstuffs by heating via the Maillard reaction. Roasting or heating foods at high temperatures (eg. greater than 100-125° C., see Wal, Thermal processing and allergenicity of foods, Allergy 58: 727-729 (2003)) will result in the addition of reactive carbonyls that, we have found, may render these foods immunogenic and drive a Th2-type immune response (Chung & Champagne, J Agric Food Chem 1999, 47, 5227-31 and 2001, 49, 3911-16).

The method of the first aspect of the invention may be used to decrease the number of reactive carbonyl groups present in the foodstuff, ie the removing step. Hence in an embodiment of this aspect of the invention the antigen in which the number of reactive carbonyl groups is reduced is present in a foodstuff. A further embodiment of this aspect of the invention is wherein the foodstuff in which the number of reactive carbonyl groups is reduced is to be incorporated into processed foods, preserved foods, baby food, ready meals, or to be applied to the skin, for example skin creams, beauty products, face packs and the like.

Reactive carbonyl groups may be removed from a food antigen using, for example, hydrogenation using hydrogen and a suitable catalyst (see above) in the method of the first aspect of the invention. Such a method is compatible with food industry practice as would be appreciated by a person skilled in the art. Hence the method of this aspect of the invention can be used to reduce the immunogenicity and Th-2-biasing (allergenic) properties of any antigens present in bulk foodstuffs before the foodstuff is consumed. This may benefit both the consumer, as there would be a reduction in the immunogenicity and allergenicity of any antigens present in the foodstuff, and may benefit the manufacturer of the foodstuff, as there may be less of a requirement to label foodstuffs as potentially allergenic.

As shown in the accompanying examples, roasted and dry-roasted peanuts have increased numbers of reactive carbonyl groups compared to raw peanuts. This may indicate a role for reactive carbonyl groups in evoking an allergenic immune response, as roasting of peanuts is epidemiologically associated with peanut allergy whereas uncooked, fried or boiled peanuts are not (Bayer et al, Effects of cooking methods on peanut allergenicity, J. Allergy Clin. Immunol. 107: 1077-1081 (2001)). Hence in a further embodiment of this aspect of the invention the foodstuff in which the number of reactive carbonyl groups is reduced is roasted nuts, for example roasted and dry-roasted peanuts.

Antigens may also be self-proteins or antigens of humans or animals. In relation to the skin, suitable antigens may include those mentioned in the following exemplary documents: Svedman et al, Deodorants: an experimental provocation study with hydroxycitronellal, Contact Dermatitis 48(8):217-223 (2003); Niwa et al, Protein oxidative damage in the stratum corneum: evidence for a link between environmental oxidants and the changing prevalence of nature of atopic dermatitis in Japan, British J Dermatol, 149:248-254 (2003). Respiratory system antigens are discussed in, for example, Rumchev et al, Domestic exposure to formaldehyde significantly increases the risk of asthma in young children, Eur Respir J, 20(2): 403-408 (2002. Liver or pancreas antigens are discussed in, for example, Tuma D J, Role of malondialdehyde-acetaldehyde adducts in liver injury, Free Radic Biol Med, 32(4):303-8 (2002); Nordback et al, The role of acetaldehyde in the pathogenesis of acute alcoholic pancreatitis, Ann Surg, 214(6):671-678 (1991), in which self protein adducted with aldehydes could potentially trigger or contribute to the hypersensitivity reactions seen. Examples of antigens include thyroglobulin, insulin, tumour specific antigens or tumour markers or DNA. The antigen may also be xenografts such as glutaraldehyde-treated heart valves of porcine and bovine origin that are used in humans (see: Salgaller and Bajpai, Immunogenicity of glutaraldehyde-treated bovine pericardial tissue xenografts in rabbits, J. Biomedical Materials Research 19: 1-12 (1985)).

As outlined above, a number of reducing agents, for example NaCNBH₃, NaBH₄, dimethylamine borane or piridine borane, can be used in the first aspect of the invention to decrease the number of reactive carbonyl groups present in the antigen. Hence a further embodiment of this aspect of the invention is wherein the decrease in the number of reactive carbonyl groups present in the antigen is effected by reduction with reducing agents. In a further embodiment of this aspect of the invention the decrease in the number of reactive carbonyl groups present in the antigen is effected by the use of hydrogenation. Also, reactive carbonyl groups may be reduced on antigens by the use of aldehyde scavenging/sequestering agents or antioxidants to treat the antigen in isolation or as therapeutic agents in vivo. Alternatively, as outlined above, there are a number of methods by which reactive carbonyl groups may be added to an antigen, for example aldehyde or formaldehyde treatment, oxidation or the Maillard reaction. Therefore, a further embodiment of this aspect of the invention wherein the increase in the number of reactive carbonyl groups present in the antigen is effected by aldehyde, including formaldehyde, oxidation or the Maillard reaction.

A second aspect of the invention provides a vaccine or vaccine component modified by the method of the first aspect of the invention.

As outlined above, the method of the first aspect of the invention may be used to decrease the number of reactive carbonyl groups present in the vaccine or vaccine component, ie the removing step. Such a modified vaccine is likely to be less allergenic than a vaccine which has not been subjected to the method of the invention. In addition, the pattern of immune response induced by the modified vaccine will lead to different patterns of protective immunity, for example leading to enhanced protection against infection at reduced vaccine dose, longer duration of vaccine protection and a lower frequency of vaccine side effects. The effect of such modification is anticipated to act on immune responses to the moiety carrying reactive carbonyl groups, and also to act on co-administered substances (for example, other components of combined multivalent vaccines). Examples of such a vaccine are provided in the accompanying examples.

As discussed above and in the accompanying examples, formaldehyde inactivation and preservation of vaccines may result in the presence of reactive carbonyl groups. Hence, in an embodiment of this aspect of the invention the vaccine or vaccine component has been chemically denatured, formaldehyde-treated or otherwise subjected to conditions causing the addition of reactive carbonyl groups prior to the reduction in the number of reactive carbonyl groups present using the method of the first aspect of the invention.

In a further embodiment of the first or second aspects of the invention, the vaccine or vaccine component modified to decrease the number of reactive carbonyl groups present is Respiratory Syncytial Virus, Measles, Influenza, human metapneumavirus, Hantavax, WEE, EEE, VEE, encephalitis viruses, anthrax, mumps, pertussis, viral hepatitis, meningitis, poliomyelitis, tuberculosis, rubella, tetanus, diptheria, coronavirus infections or other local or systemic infection of animals or man.

Alternatively, the method of the first aspect of the invention may be used to increase the number of reactive carbonyl groups present in the vaccine or vaccine component, ie the adding step. Examples of possible vaccines or vaccine components for which this may be desirable include vaccines to helminth parasites such as schistosoma and filaria, as discussed above. Hence, in an embodiment of this aspect of the invention the vaccine or vaccine component has been modified to increase the number of reactive carbonyl groups present using the method of the first aspect of the invention.

A third aspect of the invention provides a foodstuff modified by the method of the first aspect of the invention. Examples of foodstuffs which are included in this aspect of the invention are outlined above. In particular, it is preferred that the foodstuff is roasted nuts, for example roasted and dry-roasted peanuts. The presence of reactive carbonyl groups in roasted nuts is discussed in the accompanying examples.

A fourth aspect of the invention is a composition comprising an antigen modified by the method of the first aspect of the invention or a vaccine or vaccine component according to the second aspect of the invention and an adjuvant.

Most proteins are poorly immunogenic or nonimmunogenic when administered by themselves. Strong adaptive immune responses to protein antigens almost always require that the antigen be injected in a mixture with an agent known as an adjuvant. An adjuvant is any substance that enhances the immunogenicity of substances mixed with it. Adjuvants differ from protein carriers in that they generally do not form stable linkages with the immunogen, although one exception to this is the adduction of reactive carbonyls to antigens. Furthermore, adjuvants are needed primarily for initial immunisations, whereas carriers are required to elicit not only primary but also subsequent responses to haptens. Commonly used adjuvants are Freund's (complete and incomplete), mineral gels (e.g., aluminium hydroxide), surface-active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants that can be employed include MPL-TDM adjuvant (monophosphoryl Lipid A, synthetic trehalose dicorynomycolate). For example, see “Vaccine adjuvants” 2000, Ed. Derek O'Hagan, Humana Press, New Jersey.

Adjuvants can enhance immunogenicity in several different ways. First, adjuvants convert soluble protein antigens into particulate material, which is more readily ingested by antigen-presenting cells such as macrophages. For example, the antigen can be adsorbed on particles of the adjuvant (such as alum), made particulate by emulsification in mineral oils, or incorporated into the colloidal particles of ISCOMs or biodegradable synthetic beads. This enhances immunogenicity somewhat, but such adjuvants are relatively weak unless they also contain bacteria or bacterial products. Such microbial constituents are a second means by which adjuvants enhance immunogenicity, and although their exact contribution to enhancing immunogenicity is unknown, they are clearly the more important component of an adjuvant. Microbial products may signal macrophages or dendritic cells to become more effective antigen-presenting cells. One of their effects is to induce the production of inflammatory cytokines and potent local inflammatory responses; this effect is probably intrinsic to their activity in enhancing responses, but largely precludes their use in humans. A third means to achieve an adjuvant effect is to adduct a reactive carbonyl to an antigen (see above).

A fifth aspect of the invention provides a pharmaceutical composition comprising an antigen modified by the method of the first aspect of the invention, or a vaccine or vaccine component according to the second aspect of the invention, or a composition according to the fourth aspect of the invention and a pharmaceutically acceptable carrier.

Whilst it is possible for an antigen, vaccine, or composition to be administered alone, it is preferable to present it as a pharmaceutical formulation, together with one or more acceptable carriers. The carrier(s) must be “acceptable” in the sense of being compatible with the said antigen, vaccine, or composition and not deleterious to the recipients thereof. Typically, the carriers will be water or saline which will be sterile and pyrogen free.

The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

A sixth aspect of the invention is the use of a reducing agent to modify an antigen to decrease the Th2-type bias of the Th1/Th2-type immune response of an animal exposed to the antigen. As set out above, reducing agents are of particular use in decreasing the number of reactive carbonyl groups present in an antigen. Suitable reducing agents include hydrides, including NaBH₄ or NaCNBH₃. Suitable methods which may be used to decrease the number of reactive carbonyl groups present in an antigen are set out above in relation to the first aspect of the invention.

A seventh aspect of the invention is the use of an aldehyde, including formaldehyde, oxidation or the Maillard reaction to modify an antigen to increase the Th2-type bias of the Th1/Th2-type immune response of an animal exposed to the antigen. As set out above, suitable aldehydes include glycolaldehyde, acetaldehyde, malonaldehyde, formaldehyde and glutaraldehyde, and the oxidation of an antigen can be performed using, for example, NaIO₄. Suitable methods which may be used to increase the number of reactive carbonyl groups present in an antigen are set out above in relation to the first aspect of the invention.

This aspect of the invention may be of particular use in the treatment of diseases characterised by excessive Th1 responses. Patients having such diseases may be treated with an autoantigen or foreign antigen which has been modified using the first aspect of the invention to increase the number of reactive carbonyl groups present in the antigen. Such a modified antigen may induce the patient to develop a non-Th1, non-pathogenic immune response, leading to reciprocal inhibition of the pathogenic Th1 immune response. An example of such an antigen may be thyroglobulin treated with formalin to induce a non-Th1 non-pathogenic immune response.

An eighth aspect of the invention is the use of an antigen modified by the method of the first aspect of the invention or a vaccine or vaccine component according to the second aspect of the invention or a composition according to the fourth aspect of the invention or a pharmaceutical composition according to the fifth aspect of the invention in the manufacture of a medicament for the prevention or treatment of a disease. Examples of diseases which may benefit from such a medicament include those diseases in which reactive carbonyl groups are generated, which could modify self proteins and contribute to the pathology by inducing unwanted immune response to self-antigens, as outlined above. Examples include diabetes, uraemia, alcoholism, or atherosclerosis.

Further examples of diseases which may benefit from such a medicament include those diseases in which an excess Th1-type immune response is generated. Hence the addition of reactive carbonyl groups to an antigen may bias the immune response more towards as Th2-type response. Examples of such diseases include rheumatoid arthritis and other autoimmune Th1 biased pathologies. Alternatively, aldehydes or other agents that adduct aldehyde onto antigens may be injected into a local site in which a pathological Th1-type response may need to be directed towards a less-pathological Th2-type response, such as in joints of rheumatoid arthritis patients. Such a procedure may be of particular use when the antigen(s) responsible for the deleterious immune response is/are not known.

A ninth aspect of the invention is the use of an antigen modified by the method of the first aspect of the invention or a composition according to the fourth aspect of the invention or a pharmaceutical composition according to the fifth aspect of the invention in the manufacture of a medicament for use as a vaccine or vaccine component.

The vaccine or vaccine component may comprise an antigen, composition or pharmaceutical composition having been modified using the method of the first aspect of the invention to reduce the number of reactive carbonyls present, ie the removing step. Examples of such a vaccine or vaccine component include those which are set out below.

Alternatively, the vaccine or vaccine component may comprise an antigen, composition or pharmaceutical composition having been modified using the method of the first aspect of the invention to increase the number of reactive carbonyls present, ie the adding step. Examples of such a vaccine or vaccine component include those which are set out below.

A tenth aspect of the invention is the use of an antigen modified by the method of the first aspect of the invention in which the number of reactive carbonyl groups is reduced or a vaccine or vaccine component according to the second aspect of the invention in which the number of reactive carbonyl groups is reduced is or a foodstuff according to the third aspect of the invention in which the number of reactive carbonyl groups is reduced or a composition according to the fourth aspect of the invention in which the number of reactive carbonyl groups is reduced or a pharmaceutical composition according to the fifth aspect of the invention in which the number of reactive carbonyl groups is reduced is in the manufacture of a medicament for use in desensitising a patient to an antigen.

The modified antigen, vaccine or vaccine component, foodstuff or composition or pharmaceutical composition comprising a modified antigen, vaccine or vaccine component, in which there is a reduction in the number of reactive carbonyl groups present, can be used to desensitise a patients so as to, for example, decrease the clinical symptoms of a hypersensitive response. Desensitisation is a procedure in which an allergic individual is exposed to increasing doses of antigen in the hope of inhibiting their allergic reactions. It probably involves shifting the balance between Th1 and Th2 cells and thus changing the antibody and cytokine profile produced.

Desensitisation with the modified antigen, vaccine or vaccine component, foodstuff or composition or pharmaceutical composition comprising a modified antigen, vaccine or vaccine component, in which there is a reduction in the number of reactive carbonyl groups present, can be used in combination with other therapies, such as allergen-non-specific anti-IgE antibodies to deplete the patient of allergen-specific IgE antibodies as discussed in, for example, WO 99/38987.

Possible antigens which could be modified according to the first aspect of the invention for use in desensitising a patient to an antigen include the major peanut allergens Ara h I and Ara h II, as described in, for example, WO 97/24139. Other antigens include any antigen which is allergenic because of the presence of reactive carbonyl groups.

A eleventh aspect of the invention provides a kit of parts comprising an antigen and a reducing agent capable of decreasing the number of reactive carbonyl groups on the antigen, or aldehyde, formaldehyde, oxidation or an agent for catalysing the Maillard reaction to increase the number of reactive carbonyl groups on the antigen, and, optionally, and adjuvant and/or a pharmaceutically acceptable carrier.

A twelfth aspect of the invention is an antigen modified by the method of the first aspect of the invention or a vaccine or vaccine component according to the second aspect of the invention for use in medicine.

Any publications referred to herein are hereby incorporated by reference.

The invention will now be described in more detail by reference to the following non-limiting Figures and Examples.

FIG. 1. Reaction of glycolaldehyde and formaldehyde with protein. (a) glycolaldehyde-protein reaction followed by reduction. (b) Proposed formaldehyde-protein reaction followed by reduction. (c) Details of formaldehyde-modification of proteins and end products.

FIG. 2. Reactive carbonyl content measured by the DNPH colorimetric assay. 10 μM ovalbumin (OVA) was treated with 20 mM glycolaldehyde (GA) or formaldehyde (FA) in PBS at 37° C. for 3 hours. Some samples were also reduced by addition of NaCNBH₃ during the incubation with aldehyde.

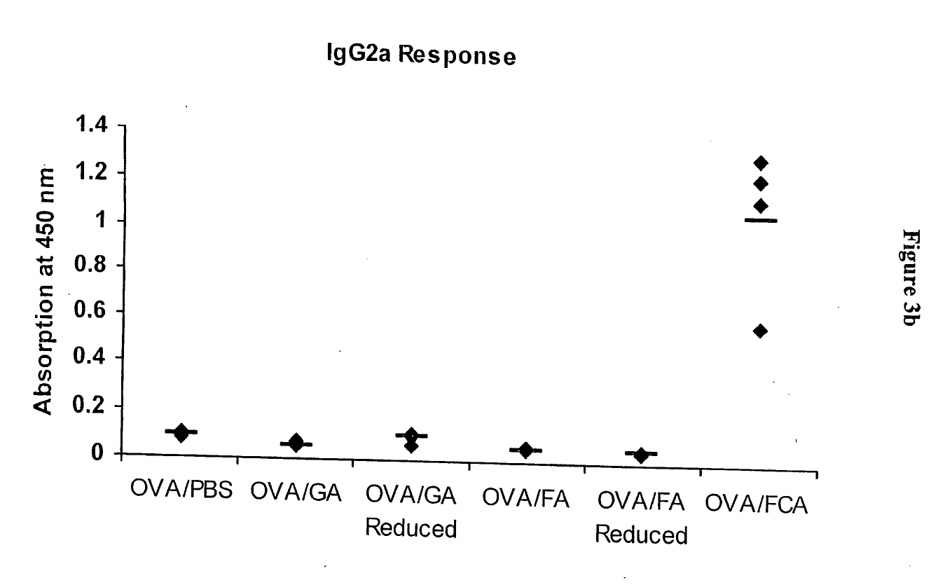

FIG. 3. Antibody responses in mice immunised with untreated OVA or OVA adducted with reactive carbonyls.

(a) IgG1 response: Mice were immunised with 25 μg OVA unmodified (OVA/PBS) or modified with glycolaldehyde (OVA/GA) or formaldehyde (OVA/FA). Modified OVA was also reduced with NaCNBH₃ to eliminate added aldehydic groups. OVA in Freund's complete adjuvant (OVA/FCA) was used as positive control. The mice were boosted with 25 μg unmodified OVA in PBS at week 4 and 2 weeks later the blood was taken and assayed for IgG1 reactivity on ELISA plate coated with native OVA. Each datum point represents the response from an individual mouse. (b) IgG2a response: carried out as (a).

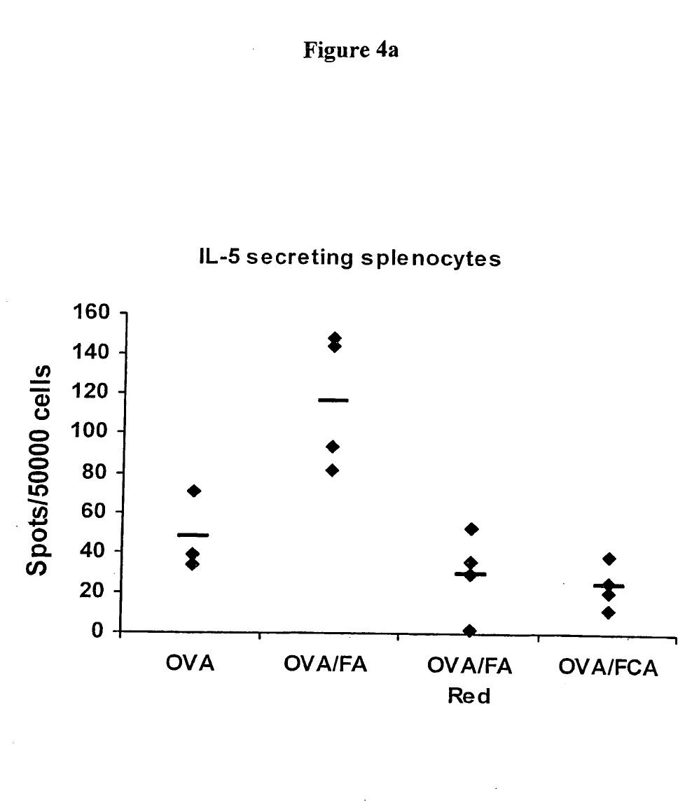

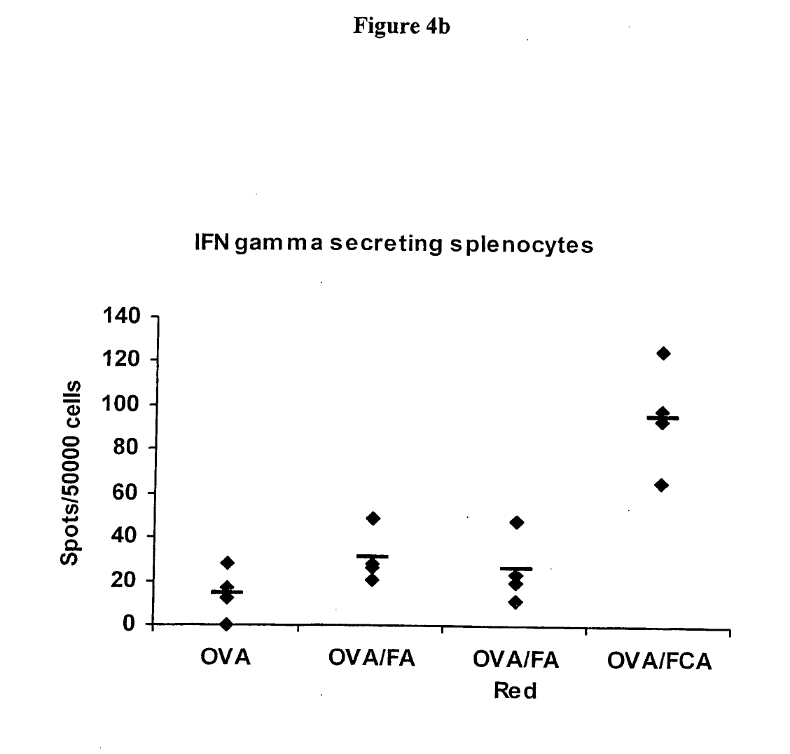

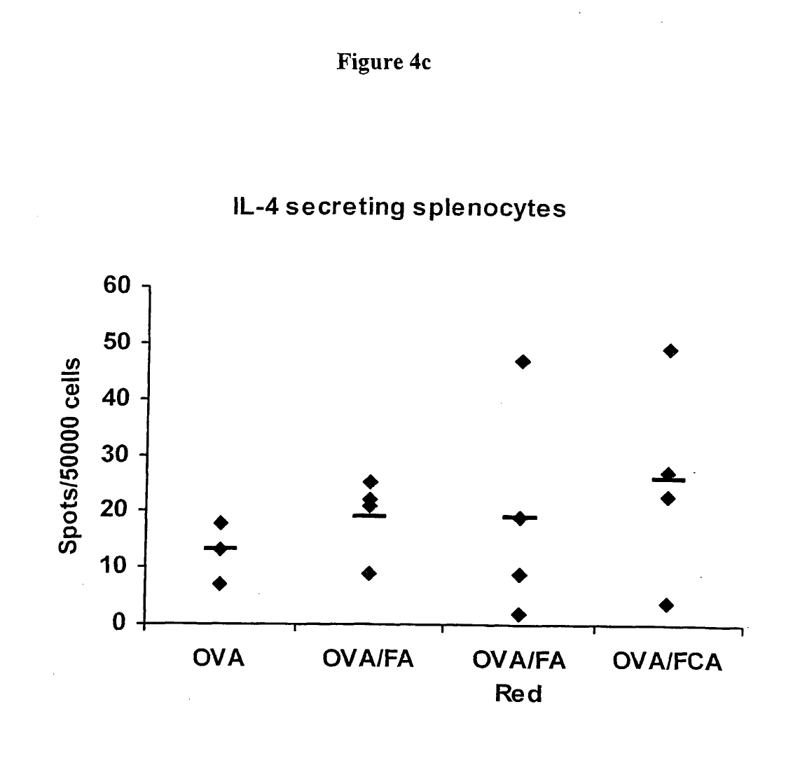

FIG. 4. Cytokine release in response to reactive carbonyl-adducted OVA.

(a) IL-5, (b) IFN-γ and (c) IL-4 producing splenocytes. Mice were immunized with OVA in PBS (OVA/PBS), or treated with 20 mM formaldehyde (OVA/FA), or treated with 20 mM formaldehyde and reduced (OVA/FA Red), or in Freund's complete adjuvant (OVA/FCA). At week 4 booster doses were administered (unmodified OVA) and 2 weeks later cells were removed from the spleens of immunised mice and pulsed with OVA in 96-wells ELISPOT plates for 24 h at 37° C. Spots were then developed and counted. Each datum point represents the response from an individual mouse.

FIG. 5. Reactive carbonyl content of RSV (respiratory syncytial virus) measured by DNPH ELISA assay. Mock infected (mock), heat-inactivated RSV (HI-RSV), formaldehyde-inactivated RSV (FI-RSV) and formaldehyde-inactivated-subsequently-reduced RSV (FI-RSV Re) were incubated with DNPH, coated onto the ELISA plates and DNPH-tagged reactive carbonyl groups were detected by an anti-DNP antibody.

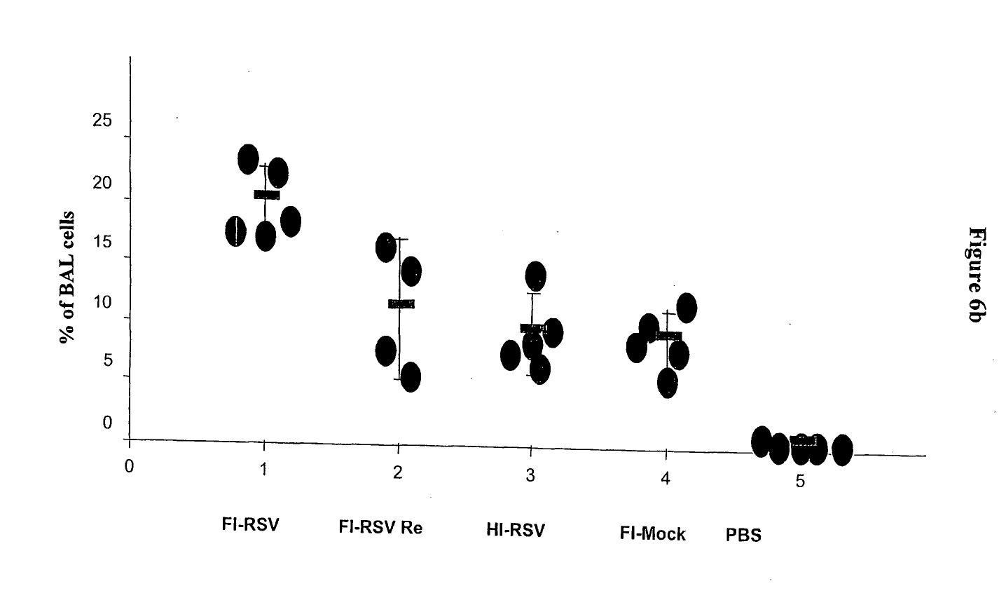

FIG. 6. Effect of FI-RSV and reduced FI-RSV vaccines on subsequent challenge with infectious RSV.

Mice were weighed daily after the live RSV challenge. (a) FI-RSV-vaccinated mice lost significantly more weight than control PBS-inoculated group and the FI-RSV-Re group for three days after the RSV challenge, whereas the HI-RSV and FI-mock groups were intermediate. (b) Eosinophils were counted in the BAL of individual mice at day 4 post challenge with live RSV. FI-RSV-Re vaccinated mice had reduced numbers of eosinophils in bronchoalveolar lavage (BAL) compared to the FI-RSV group, whereas the control group immunised with PBS alone had no detectable eosinophils. The HI-RSV and FI-mock groups had intermediate numbers of eosinophils. (c) CD8+ T cells were counted in BAL from individual mice at day 4 post-challenge with live RSV. Significantly higher CD8+ T cell numbers were present in BAL from mice immunised with FI-RSV-Re and HI-RSV when compared to FI-RSV-immunised mice. FI-mock and control PBS groups had an intermediate number of CD8+ T cells.

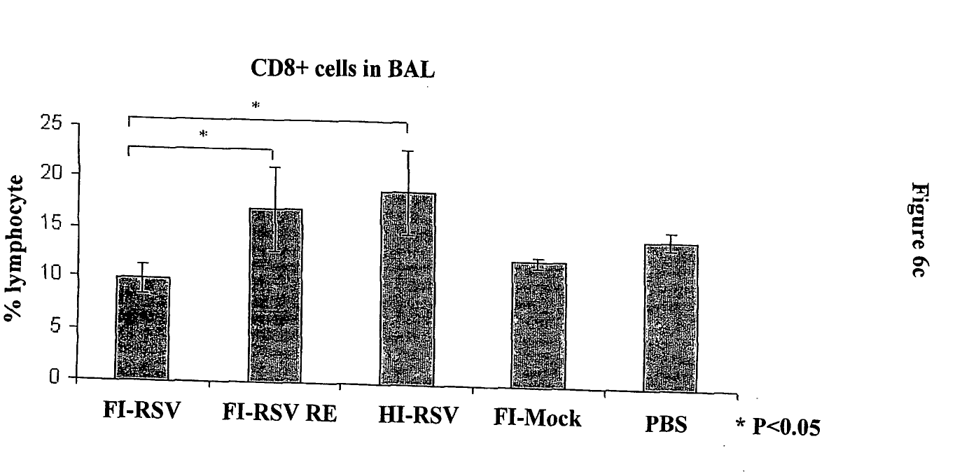

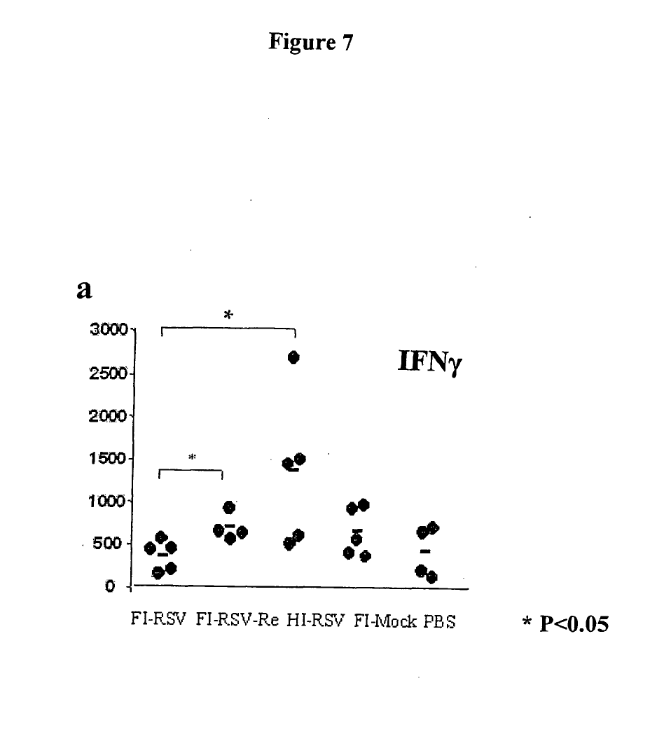

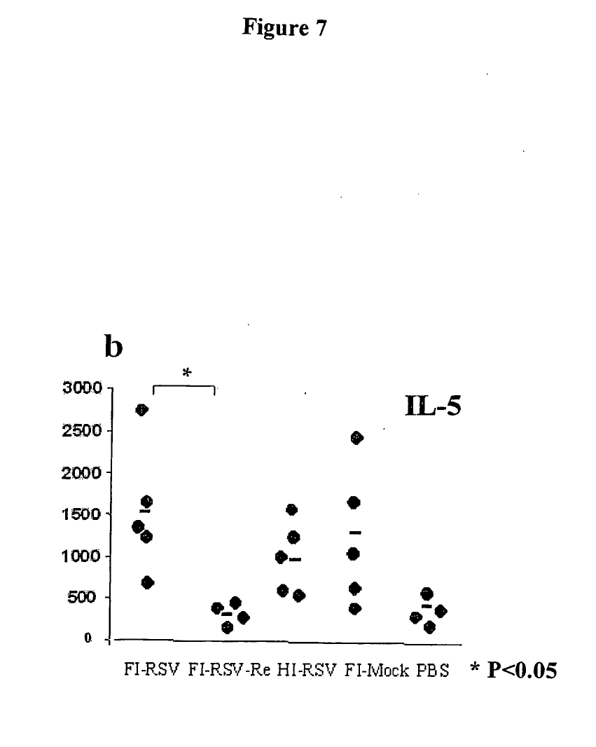

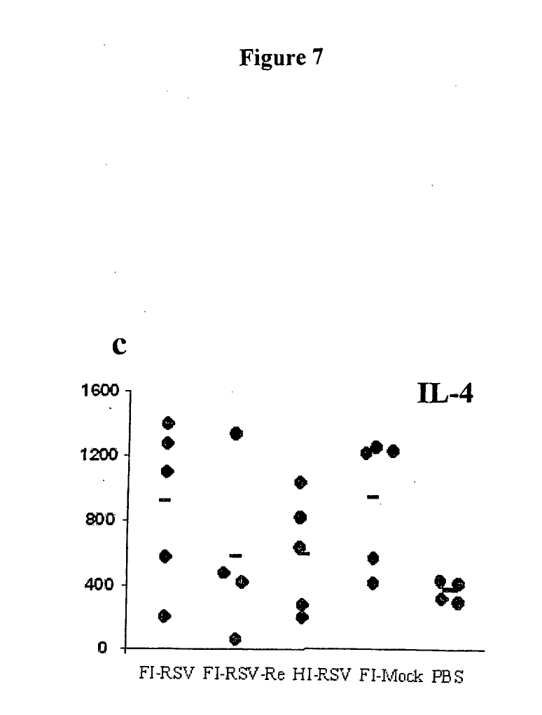

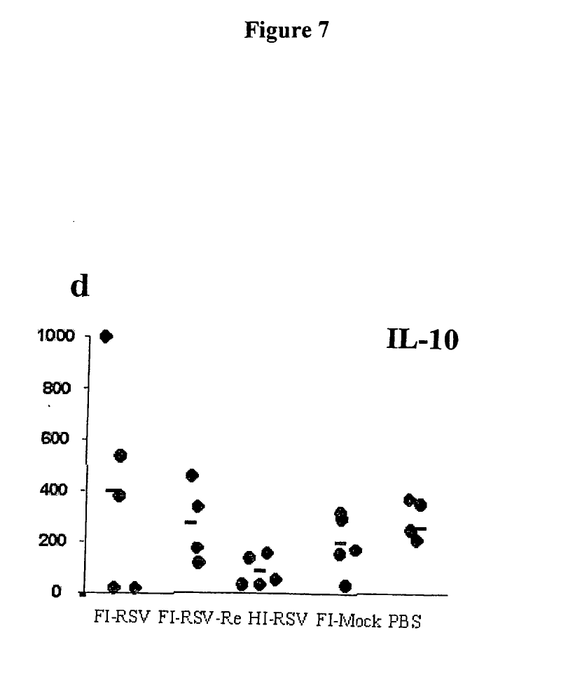

FIG. 7. Cytokine production by lung cells. Cytokines were measured by ELISPOT on day 4 after the RSV challenge. Results are expressed as number of cell producing a cytokine per one million cells. (a) the number of IFN-γ-secreting T cells was significantly higher in the FI-RSV-reduced and the HI-RSV groups than the FI-RSV group. The FI-mock group was intermediate and the PBS control group was similar to the FI-RSV group. (b) The number of IL-5-secreting T cells was significantly higher in the FI-RSV group than in the FI-RSV-reduced group. The HI-RSV and FI-mock groups had intermediate numbers of IL-5 secreting T cells and the PBS control group was similar to the FI-RSV-Reduced group. (c) The number of IL-4-secreting T cells was higher in the FI-RSV than the FI-RSV-reduced group. The FI-mock group was similar to the FI-RSV group whereas the HI-RSV group was similar to the FI-RSV-reduced group. The PBS control group had the lowest number of IL-4-secreting T cells. (d) The FI-RSV group had the highest number of IL-10 secreting T cells: lower numbers of IL-10 secreting T cells were obtained from the FI-RSV-reduced, HI-RSV, FI-mock and PBS immunised groups.

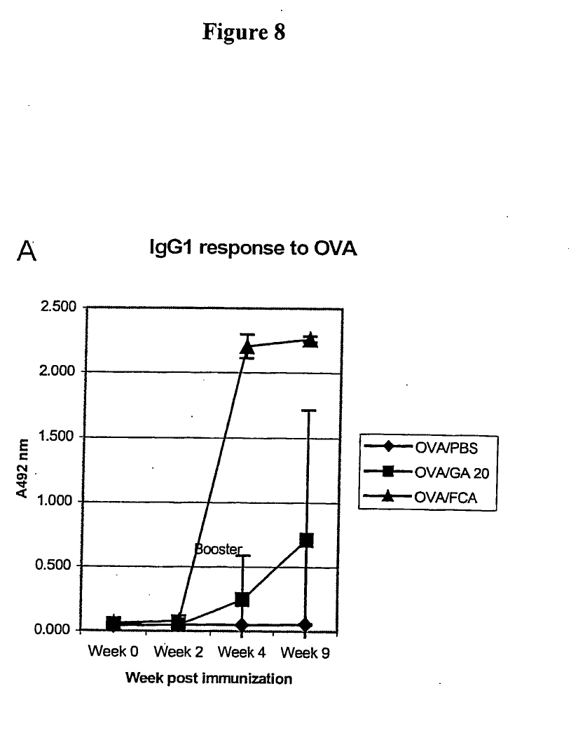

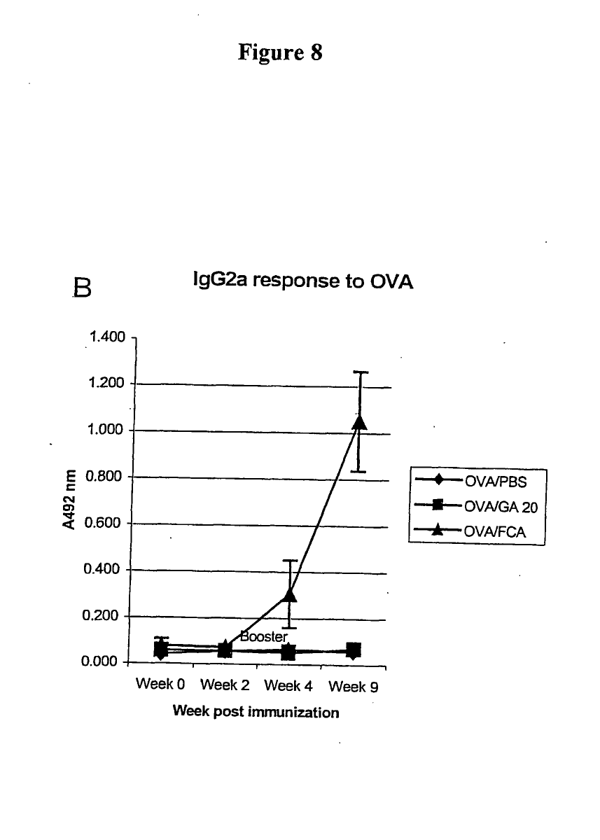

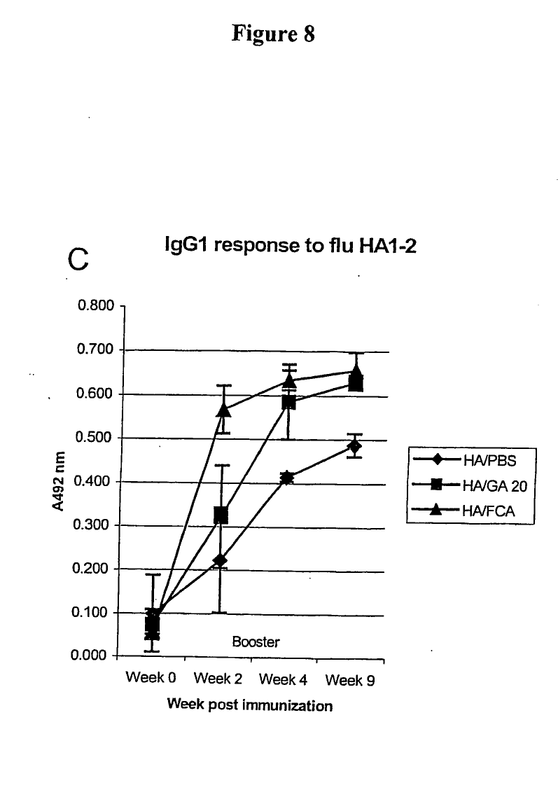

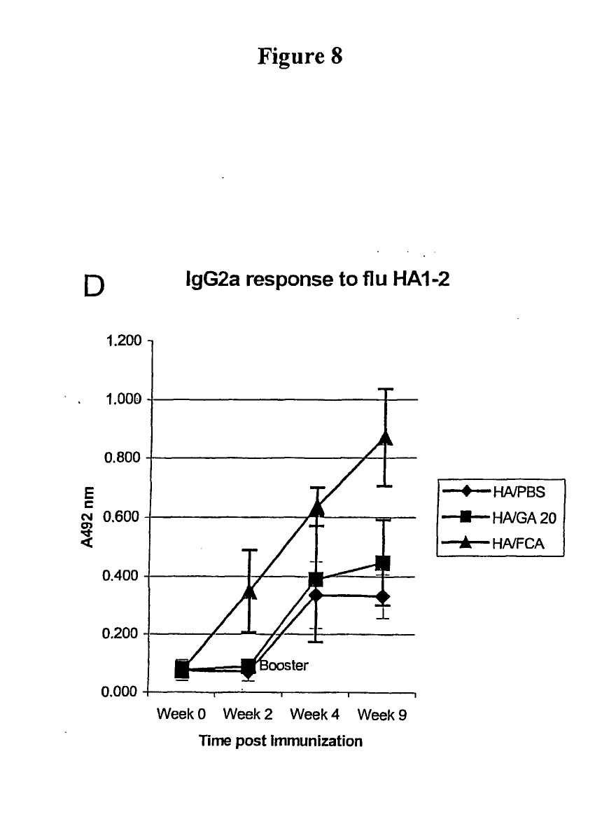

FIG. 8. Th2 antibody isotype profile of mice immunised with glycolaldehyde-treated ovalbumin or influenza haemagglutinin (HA)

A-D: Female CBA mice were immunised via the subcutaneous route with 25 μg of ovalbumin (A-B) or influenza HA (C-D) in PBS, modified with 20 mM glycolaldehyde (GA), or mixed with Freund's complete Adjuvant. The mice were boosted with native unmodified protein in PBS at week 3 post-immunisation. Sera diluted 1/100 was assayed for specific IgG1 and IgG2a on ELISA plates coated with OVA or HA and detected by anti-mouse IgG1 or IgG2a-HRP conjugated antibodies. Error bars represent ±1 standard deviation of the mean values obtained from four mice in each group. (A) shows the IgG1 response to OVA either unmodified, modified with 20 mM glycolaldehyde, or mixed with FCA. (B) shows the IgG2a response to OVA treated as in (A). (C) shows the IgG1 response of influenza HA treated as for OVA in (A), and (D) shows the IgG2a response to HA treated as for OVA in (A).

FIG. 9: Quantification of reactive carbonyl adduction to OVA using the colorimetric DNPH assay. OVA was untreated (OVA/PBS) or treated with 2 mM, 10 mM or 20 mM of glycolaldehyde (GA), or subsequently reduced with NaBH₄ at 10 mM or 100 mM as described above, then reactive carbonyls were measured using the colorimetric DNPH assay described below.

FIG. 10: Reactive carbonyl contents of commercial raw or roasted peanut protein extracts and their reductive elimination

Peanut proteins were extracted and solubilised as described in the protocol (see below) and the concentration assayed by BCA protein assay. The samples were reduced in the presence of 0.1M NaBH₄ for 2 h at 37° C. and then desalted using a Microcon 10. An ELISA to measure reactive carbonyl groups on the peanut proteins was carried out as described (above) using 5 μg protein/well.

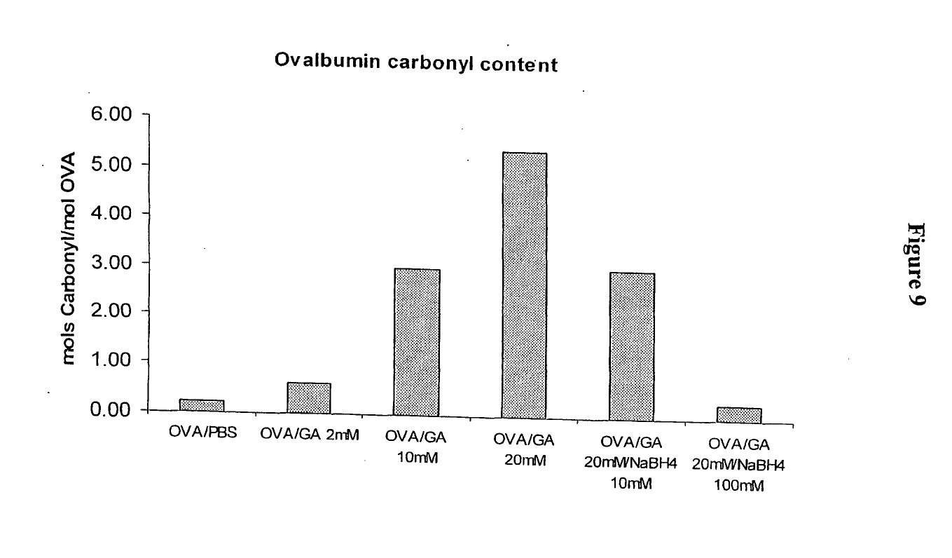

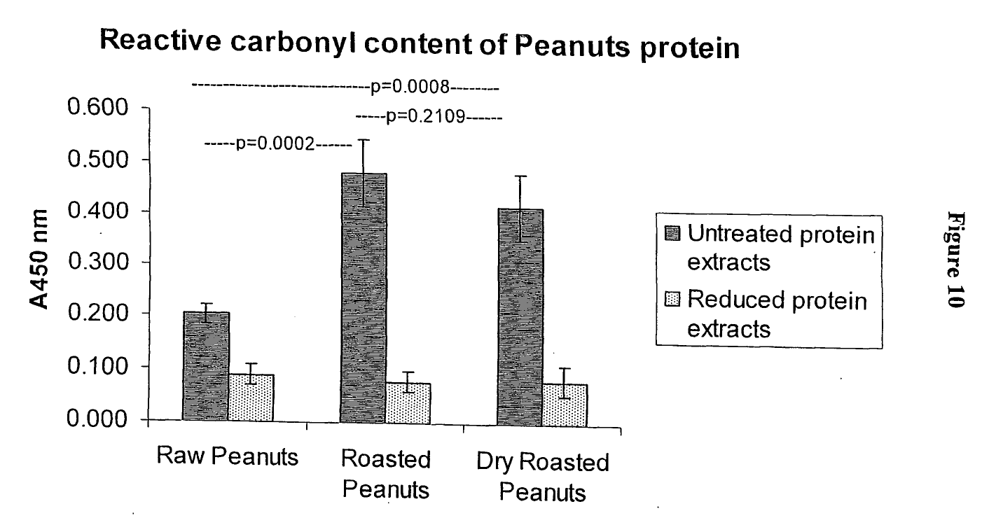

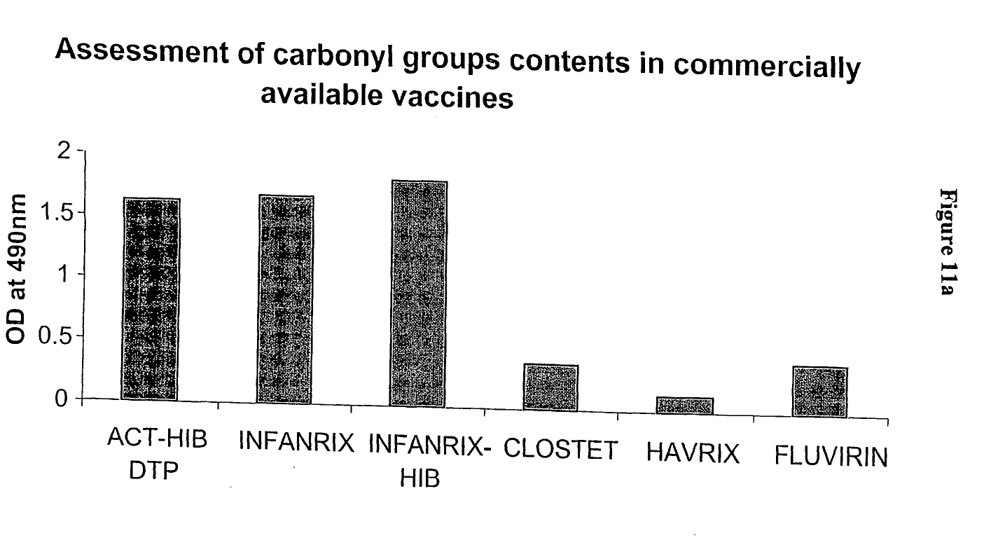

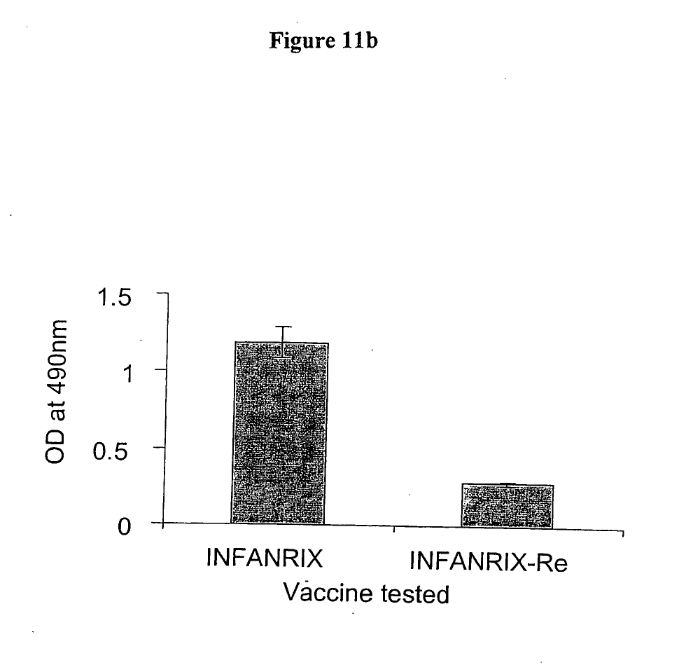

FIG. 11. Reactive carbonyl contents in commercially available vaccines.

a) Protein concentration in vaccine preparations was measured by BCA protein assay; 1 μg of protein/well was used in ELISA to measure reactive carbonyl groups by the method described above. b) One of the commercially available vaccines is shown here to contain high number of reactive carbonyls but the treatment with sodium cyanoborohydride (method described above) is able to reduce the number of these groups.

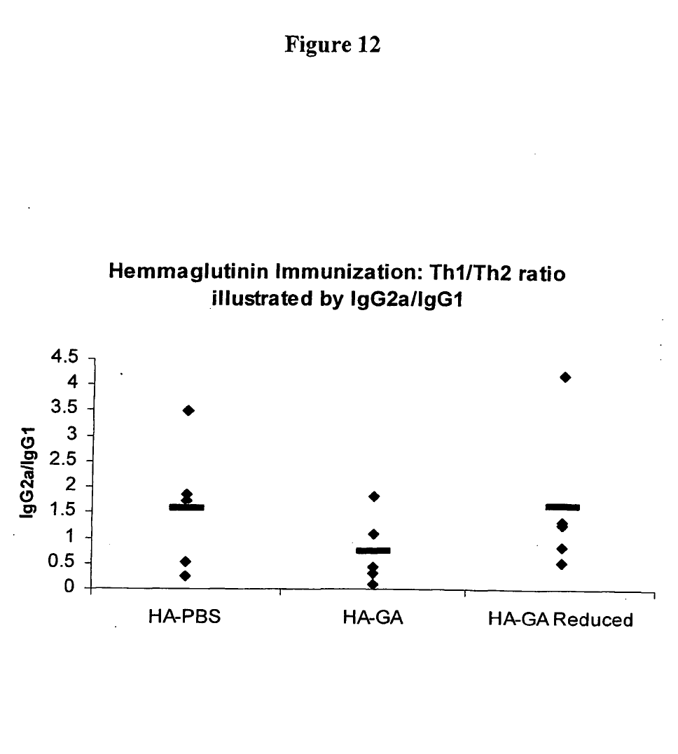

FIG. 12. Haemagglutinin Immunisation: Th1/Th2 ratio illustrated by IgG2a/IgG1

Balb/c mice were immunised with flu haemagglutinin (HA) in its native form, treated with glycolaldehyder (GA) or glycolaldehyde treated and reduced with NaBH₄. 3 weeks later mice were boosted with native HA and sera taken after a week and tested for IgG1 and IgG2a response to native HA. The ratio of IgG2a response to IgG1 was calculated as an indicator of Th1/Th2 balance.

FIG. 13. Treatment of OVA with MDA and HNE: reactive carbonyl groups

The ability of malondialdehyde (MDA) and hydroxynonenal (HNE) to add reactive carbonyl groups to OVA and reducibility of the adducts by NBH₄, assessed by DNPH ELISA.

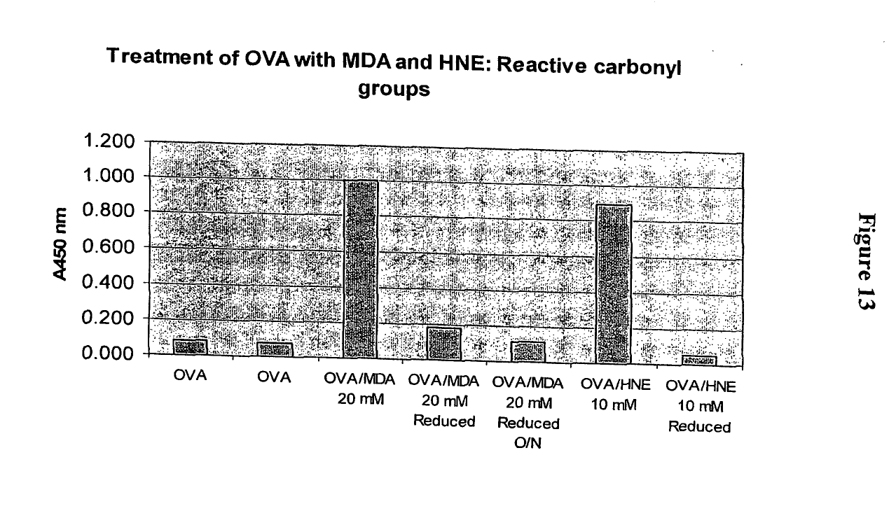

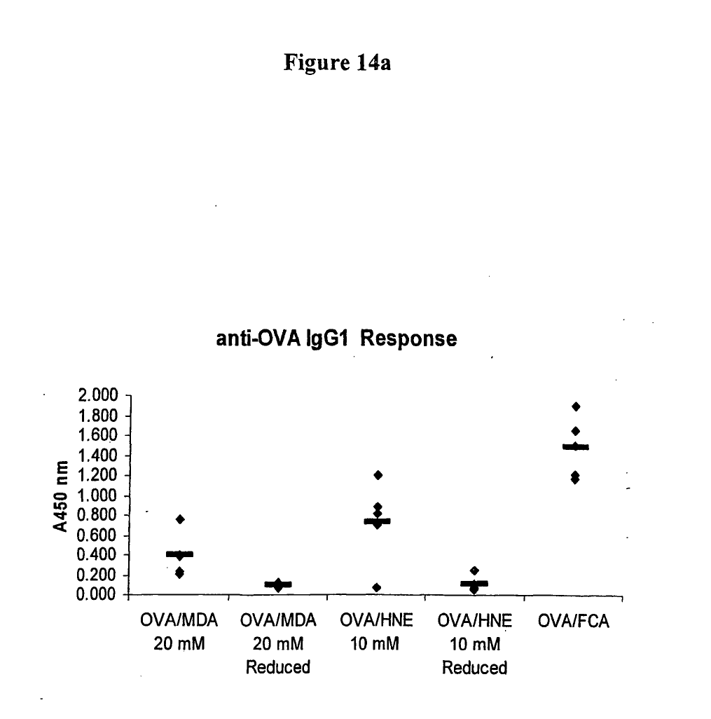

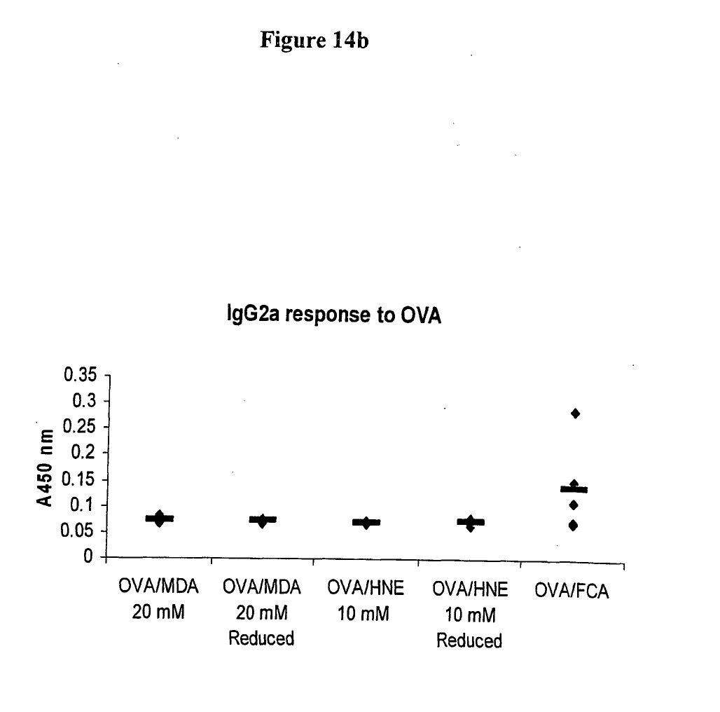

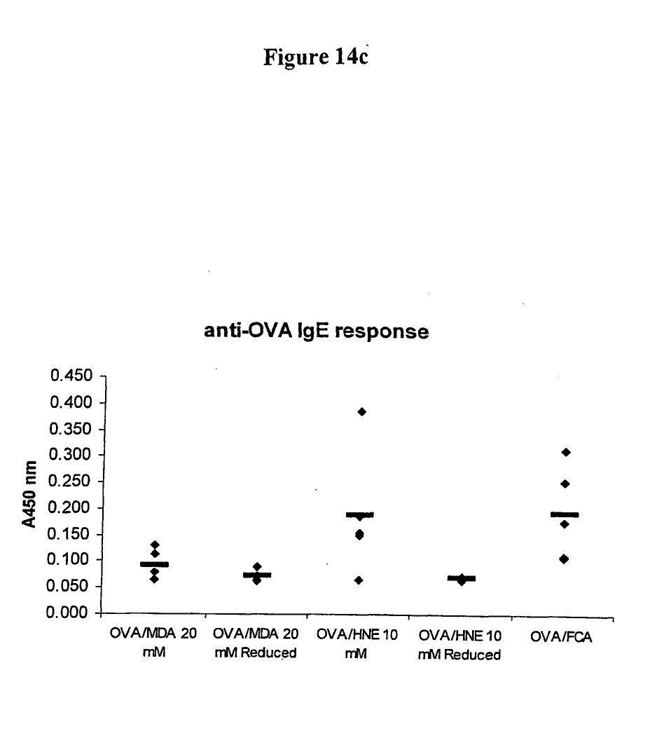

FIG. 14. Effects of immunisation with MDA and HNE-treated OVA Balb/c mice were injected s.c. with 30 μg OVA modified with either malondialdehyde (MDA) or hydroxynonenal (HNE), modified with aldehyde and reduced with NaBH₄, or in Frenudn's Complete Adjuvant (FCA). Three weeks later the animals were boosted s.c. with 30 μg unmodified OVA. Sera was taken and IgGa/IgG2a and IgE responses against unmodified OVA was detected using ELISA (IgE graph reveals the response before boosting).

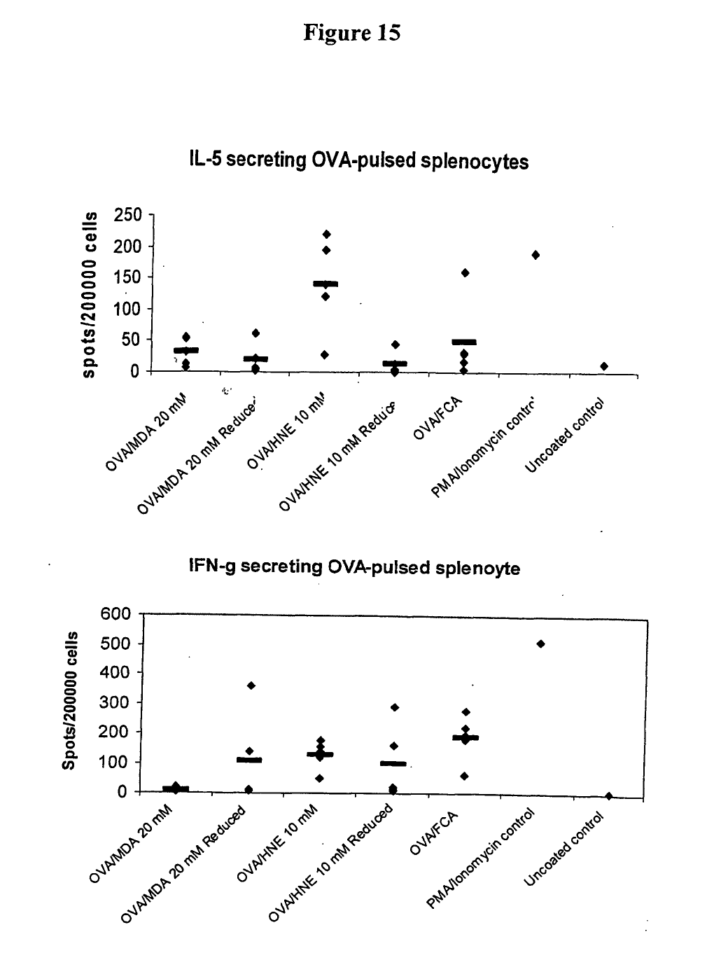

FIG. 15. Cytokine release in response to MDA and HNE-treated OVA

Splenocytes from immunised balb/c mice with MDA or HNE modified OVA, modified and reduced OVA, or OVA in FCA were stimulated in vitro with OVA and IL-5 and IFN-gamma secretion was monitored by ELISPOT.

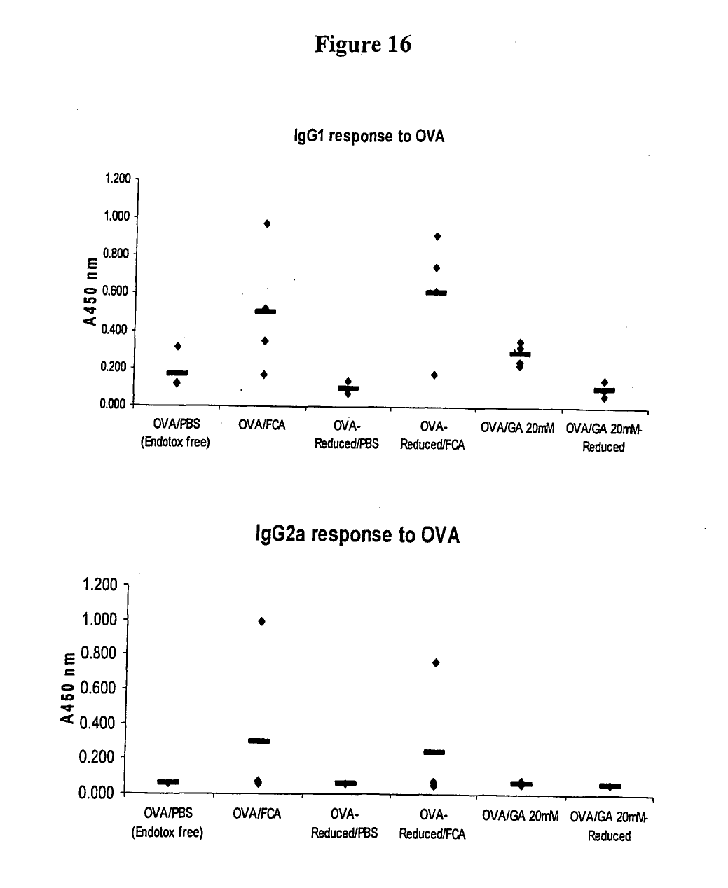

FIG. 16. Antigenicity and immunogenicity of reduced OVA

25 μg OVA was injected s.c. into balb/c mice, either untreated or reduced with NaBH₄, in PBS or FCA. Also glycolaldehyde (GA)-treated OVA and GA-treated and reduced OVA were injected in similar doses. Animals were boosted after 3 weeks with native OVA and sera assayed for IgGa and IgG2a responses to native OVA.

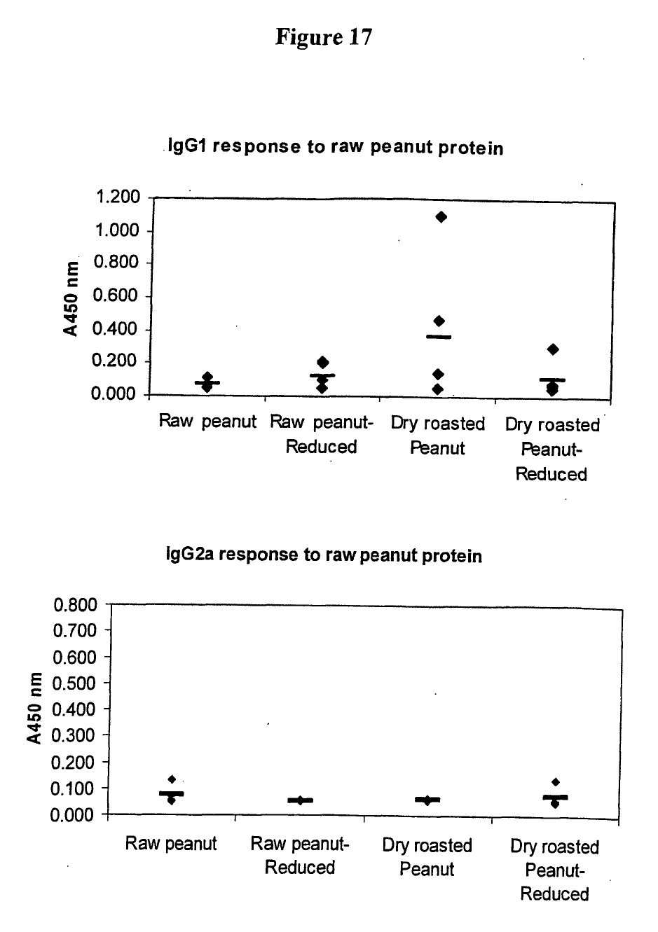

FIG. 17. Immunogenicity of roasted and raw peanut proteins

Balb/c mice were immunised s.c. with 50 μg of raw, raw-reduced with NaBH₄, dry-roasted, and dry-roasted-reduced with NaBH₄ peanut protein. 3 weeks later sera was assayed for IgG1 and IgG2a responses to raw peanut protein.

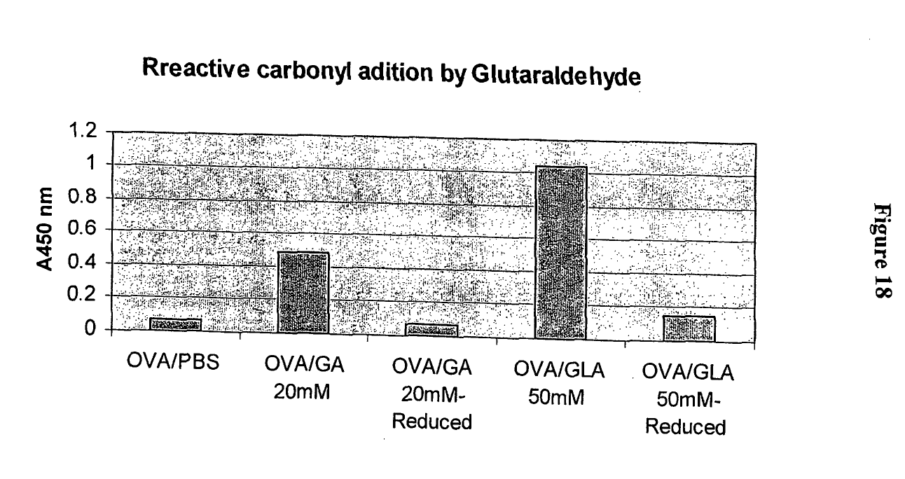

FIG. 18. Reactive carbonyl addition by Glutaraldehyde

### EXAMPLE 1

### Reactive Carbonyl Groups on Antigens Drive a Th2-Type Immune Response: a Molecular Mechanism for Hypersensitivity Reactions Elicited by Formalin-Inactivated Vaccines

Introduction

Reactive carbonyls are chemical groups that include highly reactive chemicals such as aldehydes or some less reactive structures such as ketones. Aldehydes are commonly used in medicine, research and industry because of their ability to react with various compounds to generate intra- and inter-molecular crosslinkage. Exposure to aldehydes is widespread as they are typical air pollutants, found in occupational environments (textile, paper, resins, wood composites) (1-3), utilized in disinfecting formulations (4), applied as fixatives/inactivators for cell and tissue study (5), and used in xenograft (6) and vaccine preparation (7). Reactive carbonyls occur in vivo under various conditions and can be generated during the oxidation of proteins, lipids, sugars and amino acids, and as a result of nonenzymatic glycation of proteins (8-15).

The pathological importance of reactive carbonyl-adducted proteins or lipoproteins has been extensively investigated in conditions such as diabetes, atherosclerosis, uremic syndrome, and ageing, where a state of high oxidative stress gives rise to such adducts (8, 11, 14-17). Macromolecules, including proteins and Low Density Lipoproteins (LDL), adducted with reactive carbonyls become prime targets for scavenging cells such as macrophages (8, 10, 12, 16, 18-22), and are actively taken up and degraded by these cells through scavenger receptors (10, 12, 20-23). Targeting antigens via reactive carbonyl adducts to macrophage scavenger receptors such as macrophage scavenger receptor-A (MSR-A) can drive them to present antigen to T cells (24), leading to increased immunogenicity of the antigen (25-28).

Formalin (formaldehyde) treatment has been a standard means of inactivating and preserving several microbial vaccines. Severe atypical reactions have, however, been reported in people immunized with formaldehyde-inactivated Respiratory Syncytial Virus (RSV) (29) and Measles virus (30) upon subsequent natural infection. In the case of formalin (formaldehyde)-inactivated RSV vaccine (FI-RSV) used in the 1960s, the development of an atypical exaggerated form of pulmonary disease in some vaccinated children following RSV infection, which led to some fatalities, put an end to the use of the vaccine. Subsequent investigation using animal models revealed that the exaggerated Th2 nature of the response to FI-RSV predisposed the recipients to atypical lung disease characterized by high levels of Th2 cytokines and extensive pulmonary eosinophilic infiltration (31-34). The mechanisms by which formalin-inactivated vaccines bias the immune response, however, have thus far not been elucidated.

We show here that reactive carbonyl groups added to chicken egg ovalbumin (OVA) by treatment with glycolaldehyde and formaldehyde, drive a Th2-type response to the antigen in mice, characterized by a Th2 cytokine profile and IgG1 antibody production. This response is abolished when the reactive carbonyl adducts on OVA are eliminated by chemically reducing them to non-reactive alkyl moities. We show that formaldehyde treatment of RSV adds reactive carbonyls and demonstrate, in a well-established model of RSV vaccine-induced pathology, that immunisation of mice with FI-RSV, as opposed to reduced FI-RSV or HI-RSV, induces a Th2-type response with associated pathology in mice subsequent to challenge with live RSV. We therefore propose that the addition of reactive carbonyls via formalin fixation is the major mechanism by which the RSV vaccine induced hypersensitivity in infants.

Results

Glycolaldehyde and Formaldehyde Treatment Generate Reactive Aldehyde Groups on OVA

Aldehydes react with protein via their aldehydic groups, also known as reactive carbonyl groups. Side amino groups, particularly of lysine, are prime targets of aldehydes such as glycolaldehyde for formation of Schiff base adducts (35) (FIGS. 1a and 1b). The Schiff base formed between glycolaldehyde and the side amino group of a lysine residue, for instance, goes through Amadori rearrangement and forms a reactive carbonyl group on protein (35) (FIG. 1a). It is through the generation of these reactive intermediates that protein-aldehyde adducts can react with other amino groups to form crosslinks.

Formaldehyde has not, with one exception (36), been attributed with the ability to add reactive carbonyls to proteins. Reactive carbonyls can be labelled by 2,4-dinitrophenylhydrazine (DNPH), and this provides a method for their detection and measurement on proteins (37). Using OVA as a model protein and a standard calorimetric DNPH assay to detect reactive carbonyls (38), we confirm the results of a previous study (36) showing that both glycolaldehyde and formaldehyde treatment of proteins add reactive carbonyls (FIG. 2). Under the same conditions and with equimolar amounts of aldehyde and protein, glycolaldehyde proved more efficient than formaldehyde in adding reactive carbonyls to OVA (FIG. 2). Although the basis of reactive carbonyl formation is well characterised for glycolaldehyde-protein adducts, little is known with regard to the ability of formaldehyde to create reactive carbonyls on proteins. The most likely process is that reactive carbonyls are formed through auto-oxidation of the formaldehyde-protein intermediate adducts (FIG. 1b). In addition, we confirm that reactive carbonyls formed by glycolaldehyde and formaldehyde treatment of OVA were eliminated by reductive alkylation of the aldehyde-protein adducts by a reducing agent (39) (FIG. 2).

Glycolaldehyde and Formaldehyde Treatment of OVA Renders it Immunogenic in the Absence of Extrinsic Adjuvants

Immunisation, in the absence of adjuvant, of BALB/c mice with OVA treated with either glycolaldehyde or formaldehyde, led to a robust IgG1 antibody response (FIG. 3a). Glycolaldehyde-treated OVA yielded higher titres of IgG1 than formaldehyde-treated OVA, in line with the higher number of reactive carbonyls added per mole of protein. Untreated OVA or treatment with glycolaldehyde or formaldehyde followed by reduction with NaCNBH₃ or NaBH₄ was non-immunogenic, whereas OVA administered in FCA elicited the highest titres. Analysis of the IgG2a isotype elicited by aldehyde-treated OVA indicated that the entire response was IgG1: no significant IgG2a response was detected (FIG. 3b). By contrast, mice immunised with OVA in FCA had a strong IgG2a response. These data demonstrate that both glycolaldehyde and formaldehyde treatment have adjuvant properties for otherwise poorly immunogenic proteins such as OVA, but demonstrate that the antibody response elicited is predominantly IgG1.

Aldehyde-Treated OVA Elicits a Th2-Type Immune Response

The predominance of an IgG1 response in aldehyde-treated OVA implied a Th2-type bias in the response. To investigate the idea that reactive carbonyl groups mediate such a bias, we studied the cytokine profile of the response to aldehyde-treated OVA in the splenocytes of the immunized mice. Splenocytes of animals that had been immunized with formaldehyde-treated OVA and subsequently stimulated in vitro with native OVA, exhibited a Th2-type cytokine release marked by an increased IL-5 (FIG. 4a) and low IFN-γ (FIG. 4b) secretion. This was in contrast to animals immunized with OVA in FCA, where a high IFN-γ and no significant IL-5 release was observed (FIGS. 4a, b). There were no significant differences in IL-4 production between the different groups of immunized animals (FIG. 4c). Aldehyde-treated OVA that was reduced had a cytokine profile similar to unmodified OVA, characterised by minimal production of IFN-γ and IL-5 (FIGS. 4a, b), consistent with its inability to elicit any IgG responses (FIGS. 4a, b).

Formalin-Inactivated RSV Contains Reactive Carbonyls

We wished to investigate whether the Th2-polarizing property of reactive carbonyl groups might apply to an aldehyde-inactivated vaccine with a historical association with a Th2 response, namely the formalin-inactivated RSV (FI-RSV) vaccine. We used a sensitive ELISA method for detection of reactive carbonyls on protein, that we had validated against the colorimetric assay using aldehyde-treated OVA. We demonstrated that the formalin-inactivated (FI)-RSV model vaccine, prepared in a manner based on the original protocol (29) contained a significantly increased number of reactive carbonyls as compared to the same material that had been heat-inactivated (HI-RSV) (FIG. 5). Predictably, we also found that the mock-infected control, which was formalin-treated, had an increased content of reactive carbonyls (FIG. 5). These are presumably associated with host cell-derived proteins in the control preparation. In accord with our findings in the OVA system, reduction of the FI-RSV material with NaCNBH₃ (FI-RSV-Red) eliminated the reactive carbonyls in the model RSV vaccine.

FI-RSV Induces a Th2-Type Immune Response in Mice Subsequent to Live RSV Challenge.

We then took advantage of a well-established murine model system that mimics the exaggerated Th2 response and corresponding lung pathology seen in FI-RSV vaccinees upon live RSV challenge (31, 34, 40). BALB/c mice were immunized with mock-infected (Mock), heat-inactivated (HI), formaldehyde-inactivated (FI), and formaldehyde-inactivated RSV that was subsequently reduced to eliminate aldehydic groups (FI-RSV-Red). All inocula were precipitated with aluminium hydroxide following the original vaccine protocol (41).

Mice were immunised twice with 50 μl of vaccine via the intramuscular route. Two weeks after the last immunisation mice were challenged intranasally with 5×10⁵ PFU of live RSV. Upon challenge, animals that had received FI-RSV vaccine had evidence of pathology demonstrated by progressive weight loss over 4 days (FIG. 6a). The mice immunised with FI-RSV developed typical lung pathology characterised by extensive lung infiltration with inflammatory cells, in particular eosinophils at day four post-challenge (FIG. 6b). By contrast, mice receiving reduced FI-RSV, HI-RSV or the formalin-inactivated mock vaccine had significantly lower numbers of infiltrating eosinophils (FIG. 6b). Higher numbers of infiltrating CD8+ T cells were observed in the BAL of reduced FI-RSV and HI-RSV than in the FI-RSV, consistent with a Th2-type bias in the mice receiving FI-RSV (FIG. 6c). In accord with this, the BAL CD8+ T cells from HI-RSV and reduced FI-RSV produced significantly higher levels of IFN-γ that those from FI-RSV immunised animals. This was confirmed by the cytokine profiles obtained from lung cells recovered from the same mice: cells from FI-RSV-immunised animals produced significantly higher levels of IL-5 and lower levels of IFN-γ than reduced FI-RSV (FIGS. 7a and b). The levels of IFN-γ and IL-5 produced by HI-RSV and mock FI-RSV were intermediate between the FI-RSV and reduced FI-RSV (FIGS. 7a and b). Although there was a trend towards higher IL-4 and IL-10 production by splenocytes in FI-RSV-immunised animals compared to reduced FI-RSV and HI-RSV, this was not significant (FIG. 7 c and d).

Commercially Available Vaccines Licensed for Human Use May Contain Reactive Carbonyl Groups.

We obtained vaccines which are available for use in humans and tested them for the reactive carbonyl content. As shown in FIG. 11a, among tested vaccines, those containing diptheria, tetanus and pertussis components (INFANRIX, INFANRIX-HIB, ACT-HIB-DTP) had highest content of reactive carbonyls. We then used sodium cyanoborohydride (method described above) to reduce these groups. Result is shown in FIG. 11b.

Discussion

We demonstrate here that the addition of reactive carbonyls to proteins by aldehydes, including formaldehyde, increases and alters their immunogenicity, skewing the immune response towards a Th2-type response in mice. We highlight one significant aspect of our finding by showing that reactive carbonyls present in an FI-RSV antigen, play a dominant role in skewing the immune response to live RSV challenge towards an exaggerated Th2 response. This aldehyde-dependent Th2 response is characterised by weight loss and extensive eosinophilic infiltration of the lung following live RSV challenge, and is selective for mice immunised with FI-RSV. Elimination of reactive carbonyl groups on the proteins in the FI-RSV vaccine, through reductive alkylation by a reducing agent, reversed the Th2-type bias and decreased the pathology. Consistent with a central role of aldehydes in the vaccine-induced pathology are the data from the HI-RSV vaccine, which contains only very low levels of aldehyde adducts and lacks many of the pathological aberrations seen upon live viral challenge in animals immunized with FI-RSV. The FI-RSV vaccine antigen used here was prepared and administered in the same manner as the original vaccine that had caused a Th2-biased atypical form of the disease upon RSV infection in immunised children. We therefore propose that the reactive carbonyls added to the original RSV vaccine by formaldehyde treatment are the major cause of the hypersensitivity associated with immunization with the original RSV vaccine. Moreover, we propose that the reductive elimination of aldehydic adducts provides a strategy for preventing such exaggerated responses in this, and other, formalin-inactivated vaccine preparations.

We consider that the high content of reactive carbonyls in some commercially available vaccines for use in human may affect immune responses. Among tested vaccines, those containing diptheria, tetanus and pertussis antigens (DTP) were shown to have highest contents of reactive carbonyls. It should be noted that toxoids routinely used in vaccines are prepared by formalin inactivation. The differential influence on immune responses to toxoids obtained either by chemical (formalin) treatment or genetic detoxication was shown in article published by Tonon et al (47). Moreover, DTP vaccines are administered three times to very young children, starting from 3^{rd} month of their live. The neonatal immune system is biased toward Th2 immune responses and one can speculate that using Th2 type immunogens may also bias later immune responses.

The immune-potentiating properties of reactive carbonyl adduction to antigen has been described recently (28). Whilst exploring the immunological adjuvant properties of aldehyde-bearing antigens, we found that aldehyde treatment of OVA elicits IgG1 but not IgG2a antibody responses. This was in contrast to OVA administered in FCA, a potent Th1 adjuvant, that elicited both IgG2a and IgG1 responses. This failure of aldehyde-adducted OVA to elicit IgG2a, which in mice is an indicator of IFN-γ production hence a Th1 response, agreed with two previous reports on increased IgG1 antibody titres elicited by aldehyde-protein adducts (28, 42). Interestingly in the latter study, the aldehyde-adduction of antigen decreased the titres of IgG2a elicited by FCA, indicating a potent skewing effect by aldehyde adducts towards a Th2-type antibody response. The data that we have obtained on cytokine expression after immunisation of mice with either aldehyde-adducted OVA or FI-RSV are in complete accord with the notion of aldehyde adduction driving a Th2-type immune response.

Although we do not yet understand how aldehydic groups skew the immune response to the adducted antigen towards Th2, the mechanism by which aldehydic adducts increase antigen immunogenicity has been at least partially elucidated. Aldehyde addition to macromolecules such as proteins and lipoproteins has been described in various pathological conditions such as atherosclerosis (8, 11), diabetes (8, 11, 15), uremia (16) and alcohol-induced liver disease (26, 42-44) when under a state of high oxidative stress aldehydic groups are generated and adducted to host proteins. These modified proteins and lipoproteins are endocytosed efficiently by macrophages through their scavenger receptors and prime T-cell-dependent humoral responses. The evidence for the central role of aldehyde-adduction in this process is compelling, and includes the following examples: a) aldehydic groups generated on glycoproteins by methods other than aldehyde treatment (e.g. oxidation) will similarly enhance the immunogenicity of the glycoprotein, eliciting an antigen-specific IgG1 response (28); b) The added aldehydic groups alter only the immunogenicity of the adducted antigen and not, for example, a co-administered unmodified antigen (28); c) the reductive elimination of aldehydic groups on proteins abrogates uptake of the modified protein by macrophage scavenger receptors and eliminates their ability to elicit antibody responses (28); d) monomeric aldehyde-protein adducts are as immunogenic as the crosslinked species (28), demonstrating that cross-linking alone is not responsible for the effects observed.

Our discovery of the Th2-type immune response-promoting properties of reactive carbonyl-adducts may shed light on pathologies other than those induced by the FI-RSV vaccine. For example, aldehydes have been implicated in the exaggerated Th2-biased atypical disease immune reaction to formalin-inactivated measles vaccine and in the induction of hypersensitivity and allergic responses to environmental and occupational aldehyde exposure. Moreover, reactive carbonyls can be added to proteins in vivo during inflammatory reactions by the production of glycoaldehyde by neutrophils (13, 45). This may lead to the induction of a local Th2-type response against self-antigens, potentially leading to type-1 hypersensitivity and allergic reactions. We suggest that our elucidation of the mechanism underlying these prevalent conditions and our demonstration that reductive elimination of reactive carbonyls reduces pathology, will lead to a greater understanding of their prevention and control.

Methods

OVA Modification by Aldehydes

OVA (10 μM) was incubated with 20 mM glycolaldehyde or formaldehyde in PBS for 3 hours at 37° C. The unreacted aldehyde was removed by centrifugal filtration of the solution through Microcon®10 centrifugation filters (Amicon Ltd.) at 5000×g 3 times, totalling ˜30³ buffer exchange. The reactive carbonyl groups were reduced either by addition of 0.1 mM NaCNBH₃ at the time of aldehyde addition to OVA, or subsequently to OVA modification through incubation with 0.1 mM NaBH₄ The samples were then desalted by filtering the solution through Microcon®10 centrifugation filters (Amicon Ltd) at 5000×g 3 times.

The BCA Protein assay (Pierce) was used to determine the protein concentrations and these were confirmed by measurement of absorbance at 280 nm.

Reactive Carbonyl Measurements

A. Colorimetric method. 125-250 μg protein was incubated with 500 μl of 10 mM 2-4, dinitrophenylhydrazine (DNPH) in 2M HCl in a volume of 500 μl for 1 hour at room temperature with vortexing every 10-15 min. The mixture was centrifuged at 11.000×g for 3 min, and the supernatant was discarded. The pellet was washed 3 times with 1 ml ethanol-ethyl acetate (1:1 V/V) to remove free DNPH, each time allowing the sample to stand for 10 min before re-centrifugation. Precipitated proteins were re-dissolved in 1 ml guanidine solution for 15-30 min at 37° C. Any insoluble material was removed by centrifugation at 11,000×g for 3 min. The optical density of the supernatant was measured at 375 nm and the reactive carbonyl content was calculated using the molar absorption coefficient of 22,000 M⁻¹cm⁻¹.

B. ELISA method. 5-10 μg of aldehyde-treated or untreated protein (OVA or RSV) was incubated at room temperature with 40 μl of 10 mM DNPH in 2M HCl for 45 min, shaking every 10 min. 150 μl of coating buffer NaHCO₃ at pH 8.5 was added to the solution, 100 μl of which was used to coat the ELISA plate overnight at 4° C. Plates were then washed with PBS and blocked in 200 μl PBS/1% BSA. The DNPH tagged to the protein was detected by using a biotinylated anti-DNP antibody and HRP-conjugated streptavidin (Jackson Laboratories). The ELISA was developed using TMB substrate (Pierce Ltd.) and the result read at 450 nm after stopping with 1M H₂SO₄.

Immunizations

6-8 weeks old female CBA or BALB/c mice were immunized subcutaneously using 20-25 μg of native or modified OVA in 100 μl PBS. The mice were boosted subcutaneously with 20-25 μg of native protein in 100 μl PBS at weeks 3 to 4 post-priming. Mice were tail-bled for sera collection and sacrificed 2 weeks after the boost and splenocytes were harvested for cytokine response analysis.

ELISA for OVA or RSV-Specific Antibodies

ELISA plates (Microlon high binding, Greiner Bio-one) were coated overnight in carbonate buffer at pH 8.5 at 4° C. with native OVA. The plates were blocked for 1 hour at room temperature with 200 μl PBA/1% bovine serum albumin (BSA, Sigma Ltd.), washed ×3 in PBS and serial dilutions of antisera or pre-immune sera were added to the plate in 100 μl PBA/1% BSA. After 1 hour at room temperature, plates were washed ×3 in PBS and OVA-specific IgG1 or IgG2a isotypes were detected using anti-mouse isotype conjugated to HRP (Jackson Laboratories Co.). The signals were developed with TMB substrate, stopped with 1M H₂SO₄ and read at 450 nm.

ELISPOT Assay for Counting Cytokine Secreting Cells Specific for OVA

ELISPOT plates (Millipore Ltd.) were coated overnight at 4° C. with 100 μl of 5 μg/ml of purified anti-cytokine antibodies to IL-2, IL4, IL-5, IL-10, IFN-γ (BD-Pharmingen Ltd.) in carbonate coating buffer pH 8.5. The coating buffer was discarded and the wells washed ×1 with 200 μl blocking buffer (PBS/1% BSA). 200 μl of blocking buffer was added to each well followed by incubation for 2 hours at room temperature (RT). Blocking buffer was discarded and 5×10⁴ and 2.5×10⁴ cells were added to duplicate ELISPOT wells and the plates were incubated at 37° C. for 24 hours. Cells were then discarded and the wells washed ×2 with 200 μl/well dd H₂O allowing wells to soak for 3-5 min each time. Wells then were washed again ×3 with blocking buffer. Detection antibodies, biotinylated IL-2, IL4, IL-5, IL-10, IFN γ (BD Pharmingen Ltd.) were then diluted 1/250 in blocking buffer and 100 μl was added to each well. Plates were incubated for 2 hours at room temperature. The detection antibodies were discarded and wells washed ×5 with 200 μl blocking buffer. 100 μl extravidin-alkaline phophatase (diluted in 1/1000 in filtered PBS) was added to each well and incubated for 2 hours at room temperature. This was discarded and plates washed ×5 with 200 μl/well wash buffer. 100 μl BCIP/NTB AKP substrate was added/well and incubated until the dark spots emerged (10-20 min). The spot development was then stopped by washing the plates in tap water and plates were air-dried. The spots were counted using an ELISPOT reader.

Vaccine Preparation.

The FI-RSV vaccine was prepared as originally described in (29). In brief, RSV was grown on HEp-2 cells, flasks were frozen and thawed, cells harvested and pooled. After sonication in a water bath for 10 min, preparations were centrifuged 10 min at 1000 rpm and the supernatant collected. Formalin was added (final concentration 1:4000) for 72 hours (37° C.) and samples ultracentrifuged for one hour at 50,000×g (Beckman L8-M ultracentrifuge with SW28 rotor) at 4° C. The pellet was diluted in 1/25^{th} of the original volume in PBS and further 4-fold concentration was achieved after 30 min precipitation on alum (4 mg/ml, Imject Alum, Pierce) and centrifugation (30 min, 1000 g). HI-RSV was prepared in exactly same way but without formalin addition. FI-Mock contained non-infected HEp-2 cells treated in the same way as FI-RSV.

Cell Recovery.

Four days after the challenge with RSV mice were sacrificed and bronchoalveolar lavage (BAL) fluid, lung tissue, spleen and serum were harvested as described previously (46). Briefly, the lungs were inflated with 1 ml of 12 mM lidocaine in Eagle's MEM and placed on ice in sterile tubes. 100 μl of each sample was cytocentrifuged onto glass slides and stained with hematoxylin and eosin for eosinophil counts.

ELISPOT Assay for Counting Cytokine Secreting Cells Specific for RSV.

Cells producing IFNγ, IL-4, IL-5 and IL-10 were enumerated by ELISPOTs. Briefly, microcellulose-bottomed 96 well plates were coated overnight with capture antibodies (Pharmingen,) in carbon-bicarbonate buffer. Lung cells were added (5×10⁴ per well) in duplicates and incubated at 37° C., 5% CO₂ for three days. Then appropriate biotinylated ant-cytokine antibody (Pharmingen,) was added for further two hours, followed by alkaline phosphatase-conjugated avidin (Sigma) and BCIP/NBT substrate (Sigma) until blue spots emerged. Spots were counted using automated spot counter (AID EliSpot Reader System). Results are presented as number of spots per one million cells.

Peanut Protein Extraction

Raw and roasted peanuts were ground by coffee-grinder and then by mortar and pestle. They were then defatted by ×3 washes with 5 volume cold acetone spun at 4000 g each time and finally dried overnight at 4° C. Protein was consequently extracted from the dried powder; powder was added to PBS and then agitated for 4 hours at room temperature. The samples were then spun at 2000 g at for 5 min at room temperature, supernatant collected and spun at 12000 g for 1 min, supernatant collected and filtered through a 0.45 μm filter. Samples were assayed for protein concentration by BCA protein assay.

### REFERENCES

1. Hultengren, M., L. Axelsson, S. Johnsson, and G. Rosen. 1992. Industrial surface coating of wood and associated health hazards. Exposure to formaldehyde and solvents during surface coating of wood with acids curing paints and laquers. Arbete Och Halsa 235:101-109.
2. Kim, C. W., J. S. Song, Y. S. Ahn, S. H. Park, J. W. Park, J. H. Noh, and C. S. Hong. 2001. Occupational asthma due to formaldehyde. Yonsei Med J 42:440-445.
3. Sadakane, K., H. Takano, T. Ichinose, R. Yanagisawa, and T. Shibamoto. 2002. Formaldehyde enhances mite allergen-induced eosinophilic inflammation in the murine airway. J. Envioron. pathol. Toxicol. Oncol. 21:267-276.
4. Kiec-Swierczynska, M., B. Krecisz, B. Krysiak, E. Kuchowicz, and K. Rydzynski. 1998. Occupational allergy to aldehydes in health care workers. Clinical observations. Int. J. Occ. Med. Env. Health 11:349-358.
5. Kiernan, J. 2000. Formaldehyde, formalin, paraformaldehyde and glutaraldehyde: what they are and what they do. Micorscopy today 1:8-12.
6. Salgaller, M., and P. Bajpai. 1985. Immunogenicity of glutaraldehyde-treated bovine pericardial tissue xenografts in rabbits. J. Biomed. Material Res 19:1-12.
7. Osterhaus, A. D., and P. de Vries. 1992. Vaccination against acute respiratory virus infections and measles in man. Immunobiology 184:180-192.
8. Ulrich, P., and A. Cerami. 2001. Protein glycation, diabetes and ageing. Recent Prog. Hormone Res. 56:1-21.
9. Miyata, T., K. Kurokawa, and C. Van Ypersele De Strihou. 2000. Advanced glycation and lipoxidation end products: role of reactive carbonyl compounds generated during carbohydrate and lipid metabolism. J. Am. Soc. Nephrol. 11:1744-1752.
10. Nagai, R., K. Matsumoto, X. Ling, H. Suzuki, T. Araki, and S. Horiuchi. 2000. Glycolaldehyde, a reactive intermediate for advanced glycation end products, plays an important role in the generation of an active ligand for the macrophage scavenger receptor. Diabetes 49:1714-1723.
11. Dean, R., S. Fu, R. Stocker, and M. Davies. 1997. Biochemistry and pathology of radical-mediated protein oxidation. Biochem J. 324:1-18.
12. Kawamura, M., J. Heineke, and A. Chait. 2000. Increased uptake of (-hydroxy aldehyde-modified low density lipoprotein by macrophage scavanger receptors. J. Lipid. Res. 41:1054-1059.
13. Anderson, M., S. Hazen, F. Hsu, and J. Heinecke. 1997. Human neutrophils employ the myeloperoxidase-hydrogen peeroxide-chloride system to convert hydroxy-amino acids to glycolaldehyde, 2-hydroxypropanal and acrolein: a mechanism for the generation of highly reactive-hydroxy and a, e-unsaturated aldehydes by phagocytes at sites of inflammation. J. Clin. Invest. 99:424-432.
14. Adams, S., P. Green, R. Claxton, S. Simcox, M. V. Williams, K. Walsh, and C. Leeuwenburgh. 2001. Reactive carbonyl formation by oxidative and non-oxidative pathways. Front Biosci 6:A17-24.
15. Glomb, M., and V. Monnier. 1999. Mechanism of protein modification by glyoxal and glycolaldehyde, reactive intermediates of the Maillard reaction. J. Biol. Chem. 270:10017-10026.
16. Miyata, T., C. Van Ypersele De Strihou, K. Kurokawa, and J. Baynes. 1999. Alteration in nonenzymatic biochemistry in uremia: origin and significance of “carbonyl stress” in long-term uremic complications. Kidney Int. 55:389-399.
17. Nicoletti, A. 1999. The macrophage scavanger receptor type A directs modified proteins to antigen presentation. Eur. J. Immunol. 29:512-521.
18. Horiuchi, S., M. Murakami, K. Takata, and Y. Morino. 1986. Scavenger receptor for aldehyde-modified proteins. J. Biol. Chem. 261:4962-4966.
19. Mattana, J., L. Margiloff, and P. Singhal. 1995. Oxidation of mesangial matrix using a mixed function oxidase system augments adhesion of macrophages: possible role of scavanger receptor. Biochem. Biophysic Res. Comm. 212:63-69.
20. Zhou, M. 1998. Oxidative and malondialdehyde modification of low-density lipoprotein; a comparative study of binding and degradation by macrophages and endothelial cells. Brit. J. Biomed. Sci. 55:192-198.
21. Sano, H., R. Nagai, K. Matsumoto, and S. Horiuchi. 1999. receptors for protein modified by advanced glycation end products (AGE)—their functional role in atherosclerosis. Mech. Ageing Dev. 107:1999.
22. Eskild, W., G. M. Kindberg, B. Smedsrod, R. Blomhoff, K. R. Norum, and T. Berg. 1989. Intracellular transport of formaldehyde-treated serum albumin in liver endothelial cells after uptake via scavenger receptors. Biochem J 258:511-520.
23. Eskild, W., B. Smedsrod, and T. Berg. 1986. Receptor mediated endocytosis of formaldehyde treated albumin, yeast invertase and chondroitin sulfate in suspensions of rat liver endothelial cells. Int J Biochem 18:647-651.
24. Nicoletti, A., G. Caligiuri, I. Tornberg, T. Kodama, S. Stemme, and G. K. Hansson. 1999. The macrophage scavenger receptor type A directs modified proteins to antigen presentation. Eur J Immunol 29:512-521.
25. Abraham, R., A. Choudhury, S. Basu, V. Bal, and S. Rath. 1997. Disruption of T cell tolerance by directing a self antigen to macrophage-specific scavanger receptors. J. Immunol. 158:4029-4035.
26. Thiele, G. M., D. J. Tuma, M. S. Willis, J. A. Miller, T. L. McDonald, M. F. Sorrell, and L. W. Klassen. 1998. Soluble proteins modified with acetaldehyde and malondialdehyde are immunogenic in the absence of adjuvant. Alcohol Clin Exp Res 22:1731-1739.
27. Wuttge, D., M. Bruzelius, and Stemme. 1999. T cell recognition of lipid peroxidation products breaks self tolerance to self proteins. Immunology 98:273-279.
28. Allison, M., and D. fearon. 2000. Enhanced immunogenicity of aldehyde-bearing antigens: a possible link between innate and adaptive immunity. Eur. J. Immunol. 30:2881-2887.
29. Kim, H. W., J. G. Canchola, C. D. Brandt, G. Pyles, R. M. Chanock, K. Jensen, and R. H. Parrott. 1969. Respiratory syncytial virus disease in infants despite prior administration of antigenic inactivated vaccine. Am J Epidemiol 89:422-434.
30. Norrby, E. 1995. The paradigms of measles vaccinology. Curr Top Microbiol Immunol 191:167-180.
31. Connors, M., AB. Kulkarni, C. Y. Firestone, K. L. Holmes, H. C. Morse, 3rd, A. V. Sotnikov, and B. R. Murphy. 1992. Pulmonary histopathology induced by respiratory syncytial virus (RSV) challenge of formalin-inactivated RSV-immunized BALB/c mice is abrogated by depletion of CD4+ T cells. J Virol 66:7444-7451.
32. Openshaw, P., F. Culley, and W. Olszewska. 2002. Immunopathogenesis of vaccine-enhanced RSV disease. Vaccine 20:S27-S31.
33. De Swart, R. L., T. Kuiken, H. H. Timmerman, G. Amerongen Gv, B. G. Van Den Hoogen, H. W. Vos, H. J. Neijens, A. C. Andeweg, and A. D. Osterhaus. 2002. Immunization of macaques with formalin-inactivated respiratory syncytial virus (RSV) induces interleukin-13-associated hypersensitivity to subsequent RSV infection. J Virol 76:11561-11569.
34. Waris, M. E., C. Tsou, D. D. Erdman, S. R. Zaki, and L. J. Anderson. 1996. Respiratory synctial virus infection in BALB/c mice previously immunized with formalin-inactivated virus induces enhanced pulmonary inflammatory response with a predominant Th2-like cytokine pattern. J Virol 70:2852-2860.
35. Acharya, A., and J. manning. 1983. Reaction of glycolaldehyde with proteins: latent crosslinking potential of a-hydroxyaldehydes. Proc. Natl. Acad. Sci. USA 80:3590-3594.
36. Adams, D. O., T. A. Hamilton, L. D. Lauer, and J. H. Dean. 1987. The effect of formaldehyde exposure upon the mononuclear phagocyte system of mice. Toxicol Appl Pharmacol 88:165-174.
37. Levine, R. L., J. A. Williams, E. R. Stadtman, and E. Shacter. 1994. Carbonyl assays for determination of oxidatively modified proteins. Methods Enzymol 233:346-357.
38. Levine, R. 1990. Determination of carbonyl content in oxidatively modified proteins. Meth. Enzymol. 186:464-478.
39. Galembeck, F., D. S. Ryan, J. R. Whitaker, and R. E. Feeney. 1977. Reaction of proteins with formaldehyde in the presence and absence of sodium borohydride. J Agric Food Chem 25:238-245.
40. Boelen, A., A. C. Andeweg, J. Kwakkel, W. Lokhorst, T. Bestebroer, J. Dormans, and T. Kimman. 2001. Both immunisation with a formalin-inactivated respiratory syncytial virus (RSV) vaccine and a mock antigen vaccine induce severe lung pathology and a Th2 cytokine profile in RSV-challenged mice. Vaccine 19:982-991.
41. Kim, H. W., S. L. Leikin, J. Arrobio, C. D. Brandt, R. M. Chanock, and R. H. Parrott. 1976. Cell-mediated immunity to respiratory syncytial virus induced by inactivated vaccine or by infection. Pediatr Res 10:75-78.
42. Willis, M. S., L. W. Klassen, D. J. Tuma, M. F. Sorrell, and G. M. Thiele. 2002. Adduction of soluble proteins with malondialdehyde-acetaldehyde (MAA) induces antibody production and enhances T-cell proliferation. Alcohol Clin Exp Res 26:94-106.
43. Willis, M. S., G. M. Thiele, D. J. Tuma, and L. W. Klassen. 2003. T cell proliferative responses to malondialdehyde-acetaldehyde haptenated protein are scavenger receptor mediated. Int Immunopharmacol 3:1381-1399.
44. Duryee, M. J., L. W. Klassen, T. L. Freeman, M. S. Willis, D. J. Tuma, and G. M. Thiele. 2003. Chronic ethanol consumption impairs receptor-mediated endocytosis of MAA-modified albumin by liver endothelial cells. Biochem Pharmacol 66:1045-1054.
45. Hazen, S. L., F. F. Hsu, A. d'Avignon, and J. W. Heinecke. 1998. Human neutrophils employ myeloperoxidase to convert alpha-amino acids to a battery of reactive aldehydes: a pathway for aldehyde generation at sites of inflammation. Biochemistry 37:6864-6873.
46. Hussell, T., L. C. Spender, A. Georgiou, A. O'Garra, and P. J. Openshaw. 1996. Th1 and Th2 cytokine induction in pulmonary T cells during infection with respiratory syncytial virus. J Gen Virol 77 (Pt 10):2447-2455.
47. Tonon, S., S. Goriely, E. Aksoy, O. Pradier, G. Del Giudice, E. Trannoy, F. Willems, M. Goldman, and D. De Wit. 2002. Bordetella pertussis toxin induces the release of inflammatory cytokines and dendritic cell activation in whole blood: impaired responses in human newborns. Eur. J Immunol. 32:3118-3125.

### EXAMPLE 2

### Treatment of Ovalbumin by Glycolaldehyde Drives a Th2 Immune Bias

Female CBA mice were immunised subcutaneously with 25 μg of ovalbumin or flu HA in PBS, modified with 20 mM glycolaldehyde (GA), or mixed with Freund's complete Adjuvant.

The mice were boosted with native HA in PBS at week 3 post-immunization. Sera were taken from the mice, diluted 1/100 and the presence of IgG1 and IgG2a antibodies was detected using ELISA plates coated with OVA or HA. Specific antibody binding to HA was detected with anti-mouse IgG1 or IgG2a-HRP conjugated antibodies.

The methods used were the same as the methods outlined in Example 1. The results from this experiment are shown in FIG. 8.

It is clear from the data presented in FIG. 8 that mice exposed to glycolaldehyde treated OVA or HA have a greater increase in IgG1 antibody production than IgG2a antibody production, indicating that the mice develop a Th2 antibody profile

### EXAMPLE 3

Quantification of reactive carbonyl addition to OVA, and their reductive elimination. Reactive carbonyls were added to OVA using glycolaldehyde as described in the methods above. Three different concentrations of glycolaldehyde were used: 2, 10 and 20 mM (FIG. 9). A sample of OVA treated with 20 mM glycolaldehyde and containing approximately 5.5 reactive carbonyls per mole of OVA was reduced with NaBH₄ at 10 or 100 mM (FIG. 9). The reactive carbonyl content of OVA after all of these modifications was quantified using the colorimetric DNPH assay described in the methods above.

### EXAMPLE 4

### Dry Roasted Peanuts Contain Higher Numbers of Reactive Carbonyls than Uncooked Peanuts

Raw and roasted or dry-roasted peanuts were coarsely ground using a commercial coffee-grinder and then finely ground using a mortar and pestle. The powder was then de-fatted by ×3 washes with 5 volumes of cold acetone, spun at 4000×g after each acetone wash, then dried overnight at 4° C. Protein was subsequently extracted from the dried powder by adding to PBS and agitating for 4 hours at room temperature. The samples were then clarified by centrifugation at 2000×g at for 5 min at room temperature. The supernatant further clarified by centrifugation at 12000×g for 1 min. The supernatant was then collected and filtered through a 0.45 μm filter (Amicon). Samples were assayed for protein concentration by BCA protein assay.

Peanut samples were treated with 0.1M NaBH₄ for 2 h 37° C. and then desalted to reduce the reactive carbonyl content.

A DNPH ELISA to measure reactive carbonyl content was carried out as described in Example 1 using 5 μg protein/well. The error bars are standard deviations of two independent duplicate ELISA. The data is presented in FIG. 10.

As can be seen from FIG. 10, roasting or dry-roasting of peanuts significantly increases the reactive carbonyl content of the peanut proteins. Reactive carbonyls were eliminated from the proteins by reduction with 0.1M NaBH₄.

### EXAMPLE 5

### Generation of Reactive Carbonyl Groups on Model Protein (Ovalbumin) by Malondialdehyde (MDA) and (E)-4-Hydroxynonenal (HNE)

MDA and HNE are mentioned above as aldehydes generated during lipid oxidation during alcohol consumption or generation of AGE. They have been associated with increased uptake of modified proteins by macrophages and increased immunogenicity.

FIGS. 13 to 15 Show:

1. MDA and HNE add reactive carbonyl groups to protein which are reducible.
2. The immunogenicity of OVA modified by these aldehydes is increased in balb/c mice, mostly in terms of IgG1 response and not IgG2a.
3. This increase in abrogated when reactive carbonyl groups are eliminated by reduction.
4. HNE-modified OVA induces high IL-5 and low IFNγ production.
5. Points 2 and 4 suggest a Th2 biased response.
6. This response is abrogated when reactive carbonyl groups are reduced.

### EXAMPLE 6

### Antigenicity and Immunogenicity of Reduced OVA

In this example we show that chemical reduction does not alter the structure of protein. Reduced OVA is injected in Freund's Complete Adjuvant (FCA) and the antibody responses against unmodified OVA are determined. Also OVA modified by glycolaldehyde (GA), or GA-modified and reduced are checked for their immunogenicity.

FIG. 16 Shows:

1. Reduced OVA elicits antibodies against the native epitopes when injected in FCA. This confirms the structural integrity of the reduced protein.
2. GA-modified OVA is more immunogenic than OVA and this is abrogated upon further reduction to eliminate reactive carbonyl groups

### EXAMPLE 7

### Immunogenicity of Roasted Peanut Proteins as Compared to Raw Peanut Proteins

FIG. 17 Shows that:

1. Roasted peanut protein appears to be more immunogenic than raw peanut protein when it is injected s.c. in balb/c mice
2. This is mainly an IgG1 and not IgG2a response
3. The reduction of roasted peanut protein extract to eliminate the reactive carbonyl group decreases the IgG1 response.

### EXAMPLE 8

### Ability of Glutaraldehyde to Add Reactive Carbonyl Groups to Model Protein (OVA)

As noted above, glutaraldehyde (GLA) may be used for preserving Bioprosthesis. The immunogenicity of such modified tissue might be changed.

FIG. 18 Shows:

1. GLA is capable of adding reactive carbonyl groups to model protein (OVA)
2. and that they are reducible

### EXAMPLE 9

### Th1/Th2 Biasing by Glycolaldehyde Treatment of the Flu Haemagglutinin (HA)

To further show Th2 bias due to reactive carbonyl groups, here we use a virus-related protein, haemagglutinin, which is part of the current formaldehyde-modified vaccine.

FIG. 12 Shows that:
1. Generation of reactive carbonyl groups on HA seems to bias the response in mice towards a more Th2 response.
2. This bias is reversed when carbonyl groups are eliminated by reduction

### EXAMPLE 10

### Methods for Reduction Reactions

Reduction Reactions

Objectives

By the end of this section you will:
1. be able to exploit the differences in reactivity of various reducing agents (hydride vs neutral reductants) in chemoselective reductions and be able to provide a mechanistic rationale to account for their differing reactivities.
2. be able to use the inherent chirality in a substrate to control the outcome of a reduction of proximal ketones to generate selectively syn and anti 1,3- and 1,2-diols.
3. be able to rationalise the outcome of these diastereoselective reactions using well defined T.S. diagrams.
4. have gained an appreciation of the versatility of transition metals in reduction reactions.
5. have gained an appreciation of the synthetic utility of dissolving metal reductions.
6. be able to use radical chemistry for deoxygenation and reduction of halides.
   II.A Reduction of Carboxylic Acid Derivatives and Related Functionality

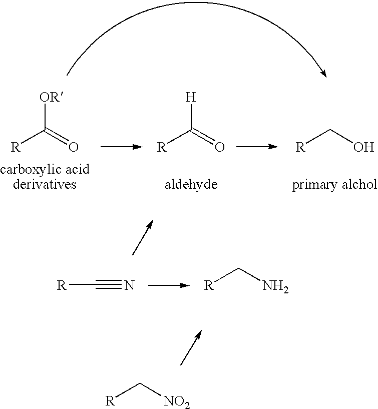

Similar issues of selectivity and reactivity to those we encountered in the case of oxidation reactions also arise in reduction reactions.
Chemoselectivity. Many different functional groups can be reduced in a variety of ways. We often need to selectively reduce one functional group whilst leaving others intact.
In the case of carboxylic acid derivatives there are two possible reduction products: aldehdye and alcohol. Ideally we need methods for selectively accessing either product.

Q? Why is it often difficult to stop the reduction of an ester at the aldehdye (consider the relative electrophilicities of the starting material and intermediate product.
Stereoselectivity. Asymmetrically substituted ketones provide secondary alcohols on reduction and introduce a new stereogenic centre into the molecule. We need methods for controlling the stereochemical outcome (relative and absolute) of this reduction using substrate or reagent (or both) control. In this course we will only consider substrate-controlled diastereoselective reductions. Enantioselective reduction is covered elsewhere (H Tye Asymmetric Synthesis course).
   II.A.1 Hydride Reducing Agents

Some of the most important reducing agents are hydrides derived from aluminium and boron. There are numerous varieties differing principally in their reactivity. They all act as sources of nucleophilic hydride and therefore are most reactive towards electrophilic species. Some of the most widely used hydride reagents are discussed below:

II.A.1.i Lithium Aluminium Hydride (LiAlH₄)

One of the most powerful reductants
Highly flammable reagent and therefore must be used with care
Reactions are normally carried out in ethereal solvents (e.g. THF, Et₂O); LiAlH₄ reacts violently with protic solvents (c.f. NaBH₄)
The extremely high reactivity of LiAlH₄ imparts relatively low levels of chemoselectivity for this reagent. However it is most reactive towards strong electrophiles.

Ease of Reduction of Some Functional Groups with LiAlH₄

| substrate | product | ease of reduction |
|---|---|---|
| aldehyde RCHO | RCH₂OH | most |
| ketone RC(O)R′ | RCH(OH)R′ | readily reduced |
| acid chloride | RCH₂OH | |
| RC(O)Cl | | |
| lactone | diol | |
| epoxide | RCH₂CH(OH)R | |
| ester RC(O)OR′ | RCH₂OH + R′OH | |
| carboxylic acid | RCH₂OH | |
| RCO₂H | | |
| carboxylate salt | RCH₂OH | |
| amide RC(O)NR′₂ | RCH₂NR′₂ | most |
| nitrile RCN | RCH₂NH₂ | difficult to reduce |
| nitro RNO₂ | RN═NR | |
| isolated alkene | | unreactive |
| RCH═CHR | | |

In addition to being capable of reducing virtually every carboxylic acid derivative, the high reactivity of LiAlH₄ makes it useful for reducing other functional groups:
Reduction of Halides and Sulfonates: 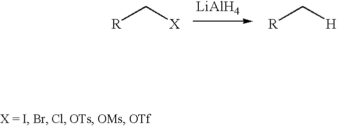
Reduction of Propargylic Alcohols to (E)-Alylic Alcohols:

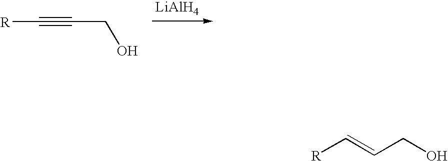

In this case the proximal alcohol is essential. The reaction proceeds through trans-selective hydrometallation of the triple bond releasing the alkene on protolytic work-up: 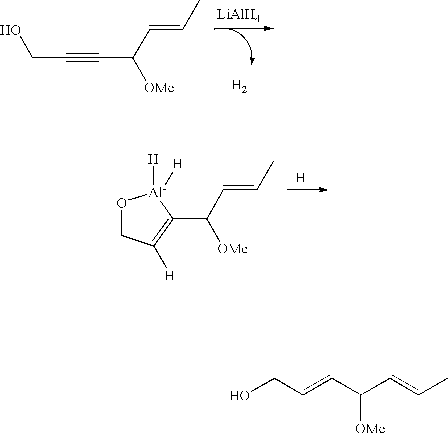
Epoxide Ring-Opening

In the case of unsymmetrically substituted epoxides issues of regioselectivity arise. In acyclic systems the nucleophile (hydride) tends to react in an S_{N}2 fashion at the less hindered end of the epoxide.

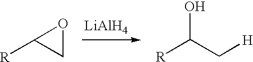

In cyclic systems there is a strong preference for axial attack (trans diaxial ring opening) 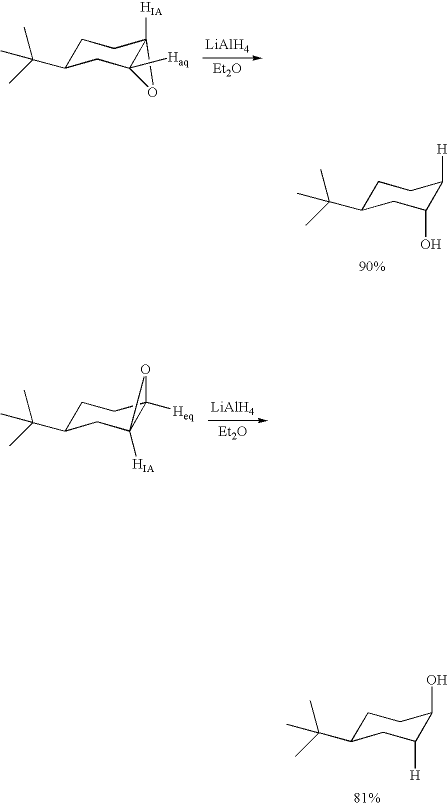
II.A.1.ii Sodium Borohydride (NaBH4)
Much milder than LiAlH₄
Frequently used to chemoselectively reduce aldehydes and ketones in the presence of esters (esters are reduced with NaBH₄ but usually at a much lower rate (less electrophilic)
reactions are carried out in protic solvents including H₂O. NaBH₄ is insoluble in most common aprotic solvents
   Related Reagents
   Lithium and Calcium Borohydride

Although the reactive component of sodium borohydride is the hydridic anion, the counterion can also be used to modulate the reactivity of the reagent system. A number of other borohydride reagents are available including LiBH₄ and Ca(BH₄)₂. Both these reagents are more reactive and readily reduce esters in addition to aldehydes and ketones. The increased reactivity of these reagents can be attributed to the increased Lewis acidity of the cations which confers increased electrophilicity on the carbonyl group (by Lewis acid-Lewis base formation).

II.A.1.iii Sodium Borohydride-Cerium (III) Chloride

Problem 1: regioselective reduction of Dr-unsaturated carbonyl groups. 1,2-reduction
good route to allylic alcohols (very important functional groups)

Solution: use a 1:1 ratio of NaBH₄ and CeCl₃—Luche Reduction

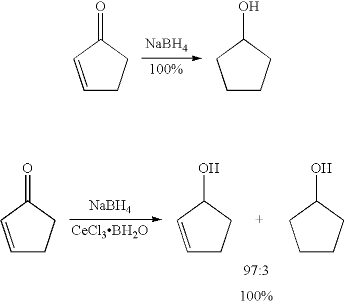

A. L. Gemal, J.-L. Luche, J. Am. Chem. Soc., 1981, 103, 5454-5459.

To obtain selective 1,4-reduction:
a) catalytic hydrogenation
b) ‘copper hydride’ [PPh3CuH]6 Stryker's reagent

Problem 2: How to chemoselectively reduce a ketone in the presence of a more electrophilic aldehyde.
Chemoselective reduction of aldehydes in the presence of ketones can usually be achieved by exploiting their increased reactivity towards nucleophilic hydride sources.

Q? Why are aldehydes more electrophilic than ketones?
Aldehydes are more electrophilic than ketones and therefore much more prone to hydration/acetalisation.
Acetals are not reduced by borohydride reagents.
Ce(III) is a good Lewis acid and strongly oxophilic—it promotes hydration of carbonyl groups especially aldehydes. Therefore it should be possible to temporarily mask an aldehyde as its acetal/hydrate to allow selective reduction of the ketone. Unmask the aldehyde in the work-up.

Solution: use 1:1 NaBH₄—CeCl₃ in wet EtOH:

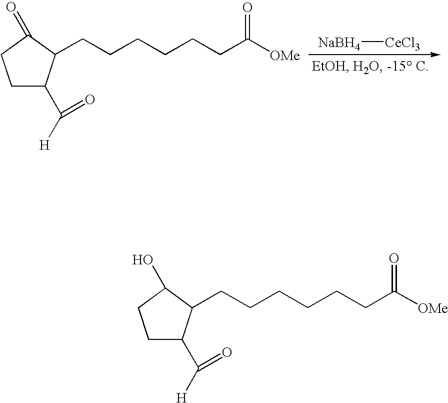

A. L. Gemal, J.-L. Luche, J. Org. Chem., 1979, 44, 4187-4189.

II.A.1.iv Sodium Cyanoborohydride (NaCNBH3)

C. F. Lane, Synthesis, 1975, 135-146.
a very useful borohydride reagent
milder than NaBH₄ at pH 7
reactivity is strongly pH dependent—it is one of the few borohydrides which tolerates acidic conditions (down to ˜pH 3)
at pH 3-4: NaCNBH3 readily reduces aldehdyes and ketones
at pH 6-7: NaCNBH3 readily reduces iminium ions but NOT C═O groups—this property is responsible for its most important use—REDUCTIVE AMINATION:
a very useful method for synthesising secondary and tertiary amines by coupling a secondary or primary amine with an aldehyde or ketone.

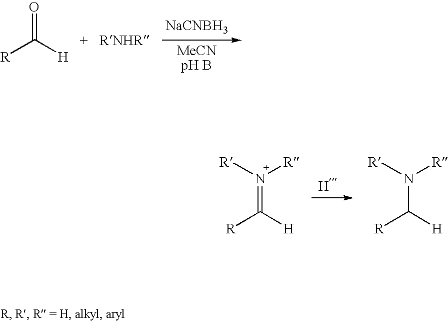

Q? An alternative method for amine formation is to alkylate a primary or secondary amine with an alkyl halide? What are the problems with this approach? Hint—is the product amine more or less nucleophilic than the starting material?

### EXAMPLE 1

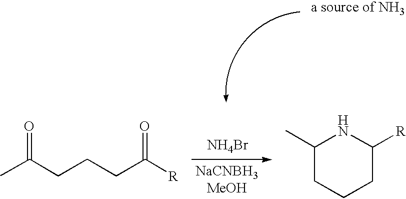

Q? Account for the stereoselectivity of this reaction.

### EXAMPLE 2

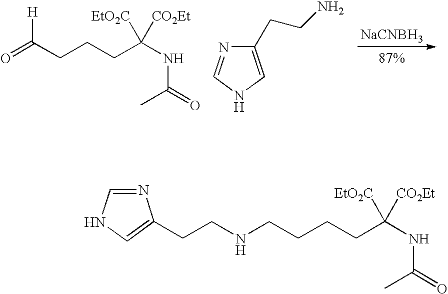 II.A.1.v Other Hydridic Reducing Agents

There are many other hydride reducing agents. The following have been developed as bulky reducing agents for use in stereoselective reduction:

| Reducing Agent | | Comment |
|---|---|---|
| LiHAl(Oi-Bu)₃ | 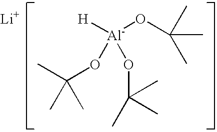 | good for converting carboxylic acid derivatives to aldehydes |
| Red-Al Na[H₂Al(OCH₂CH₂OMe)₂] | 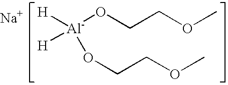 | similar reactivity to LiAlH₄ |
| L-Selectride LiHB(CH(CH₃)CH₂CH₃)₃ | 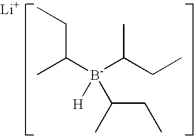 | similar reactivity to LiBH₄ |

Stereoselective Reduction of 4-Tert-Butylcyclohexanone

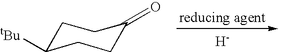

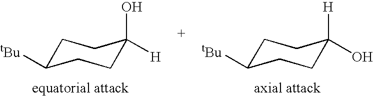

| reducing agent | equatorial attack | |
|---|---|---|
| LiAlH₄ | 10 | 90 |
| (unhindered) | | |
| LiAlH(OtBu)₃ | 10 | 90 |
| (more hindered) | | |
| LiBH(sBu)₃ (very | 93 (RT) | 7 (RT) |
| hindered) | 96.5 (−78 C.) | 3.5 (−078 C.) |
| Lithiumtrisamyl- borohydride 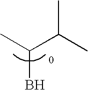 (very very hindered) | 100 | 0 |

What factors might affect the stereochemical outcome of this reduction? Hint: consider such factors as the approach trajectory of the incoming nucleophile, the size of the nucleophile. Draw Newman projections of the starting ketone and the two products and consider how the molecule reorganises on proceeding from starting material to product—eclipsing interactions are unfavourable.

II.A.2 Neutral Reducing Agents

The reagents discussed above are all hydridic and behave as nucleophiles—they react most readily with good electrophiles.

Another class of reducing agents are those which are neutral. They react through a different mechanism and as a result have quite different selectivities which are often complementary to the hydride reagents discussed earlier.
Basic Mechanism

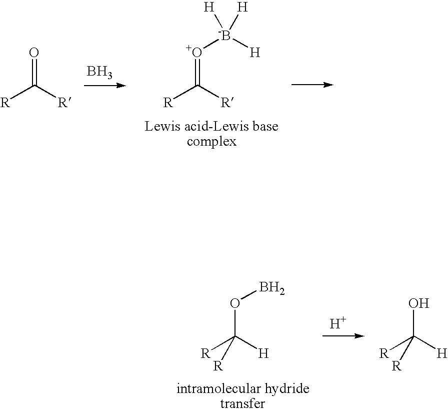

Comparison Between Borohydride and Borane

| Borohydride | Borane |
|---|---|
| negatively charged | neutral |
| nucleophilic | electrophilic |
| Valence shell of the central | 6 electrons in the valence shell |
| boron is a complete octet | of the central boron - vacant |
| | pAO confers Lewis acidicity |
| hydride transfer proceeds | hydride transfer is often |
| intermolecularly | intramolecular via a Lewis |
| | acid-Lewis base complex |

II.A.2.i Borane (BH3)

Borane is too unstable to be isolated (exists either as the dimer B₂H₆ or a Lewis acid-Lewis base complex e.g. BH₃THF or BH₃Me₂S).
very useful reagent for selectively reducing carboxylic acids to alcohols in the presence of esters
amides are also readily readily reduced to the corresponding alcohols

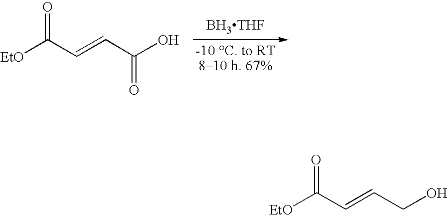

The more electron rich carboxylic acid derivatives appear to be reduced most readily—complete opposite reactivity to hydridic reducing agents.

Q? Why are carboxylic acids reduced so fast relative to esters?

Key:

borane reacts with the carboxylic acid to generate a triacyloxyborane (protonolysis). This is essentially a mixed anhydride and therefore very reactive. Esters cannot react in this way and are therefore reduced at a slower rate. 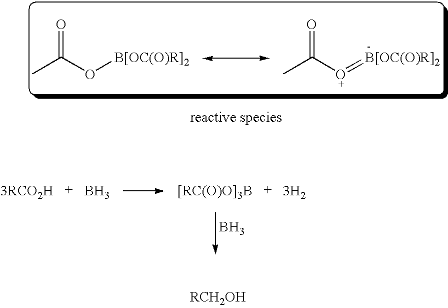
   A Note of Caution!

Borane is a good reducing agent but it is also very useful for hydroborating unsaturated systems (triple and double bonds)—chemoselectivity may be a problem.

Ease of Reduction of Some Functional Groups with Borane

| substrate | product | ease of reduction |
|---|---|---|
| carboxylic acid | RCH₂OH | most |
| RCO₂H | | readily reduced |
| isolated alkene | (RCH₂CHR)₃B | |
| RCH═CHR | | |
| ketone RC(O)R′ | RCH(OH)R′ | |
| nitrile RCN | RCH₂NH₂ | |
| Epoxide 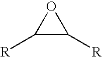 | RCH₂CH(OH)R | most difficult to reduce |
| ester RC(O)OR′ | RCH₂OH + R′OH | |
| acid chloride | | inert |
| RC(O)Cl | | |

II.A.2.ii Diisobutylaluminium Hydride (DIBALH) 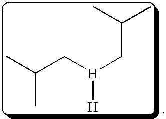
very widely used reducing agent especially for reducing esters
   esters can be reduced to either the aldehyde or the alcohol depending on the stoichiometry and reaction conditions:

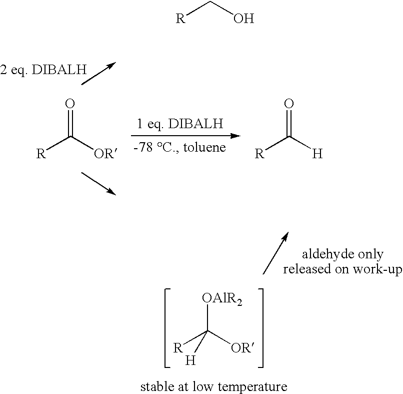

Nitriles are also reduced to aldehydes. In this case reaction proceeds via the imine which hydrolyses on acidic work-up to afford the aldehyde product:

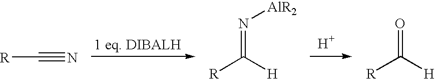

Lactones provide a useful method for preventing over-reduction of the aldehyde product. In these cases the lactone is reduced to a lactol, the hemiacetal functionality essentially masking the aldehdye and preventing over-reduction: 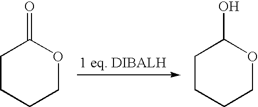
II.A.2.iii Meerwein-Ponndorf-Verley Reduction with Al(OiPr)₃
a relatively old method of reducing carbonyl groups (principally aldehydes and ketones)
isopropanol behaves as the hydride donor
the by-product is acetone
the reaction is reversible—the reverse oxidation is known as the Oppenauer Oxidation.
the mechanism is typical of a range of reagents proceeding through a well-defined chair-like T.S. (Zimmerman-Traxler) in which the beta-hydride is transferred intramolecularly to the carbonyl group.

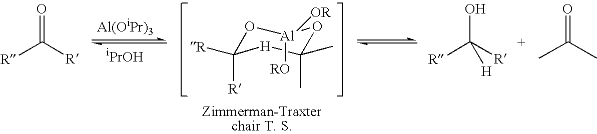

Compare this reaction mechanism with methods for directed reduction of r-hydroxyl ketones (Me₄NHB(OAc)₃— and the Evans-Tischenko reduction) later—the mechanism is very similar—CHAIR-LIKE ZIMMERMAN-TRAXLER transition states are very commonly used to rationalise the stereochemical outcome of reactions which can proceed through 6-membered transition states.

II.B Stereoselective Reduction of Prochiral Ketones

The addition of hydride nucleophile to a chiral ketones provides diastercoisomers—when the stereogenic centres are close to the carbonyl group (1,2- or 1,3-disposed (i.e. D- or r-hydroxy ketones)) then by careful choice of protecting group, reaction conditions and reducing agent a high degree of stereoselectivity can often be obtained in the reduction. 1,2- and 1,3-diols are widespread in natural products (see erythromycin and related polyketide macrolides later). Stereoselective reduction of hydroxyketones provides a reliable route to incorporating such functionality.
Diastereoselective 1,3-Reduction: 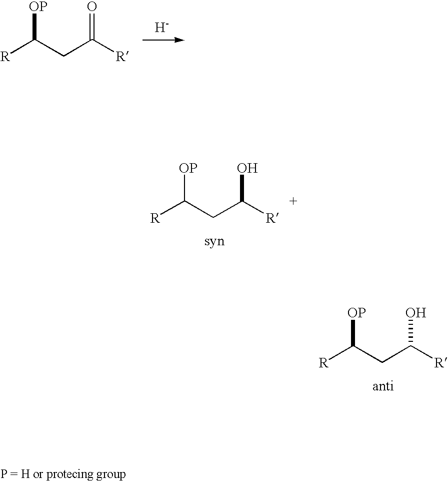
Diastereoselective 1,2-Reduction:

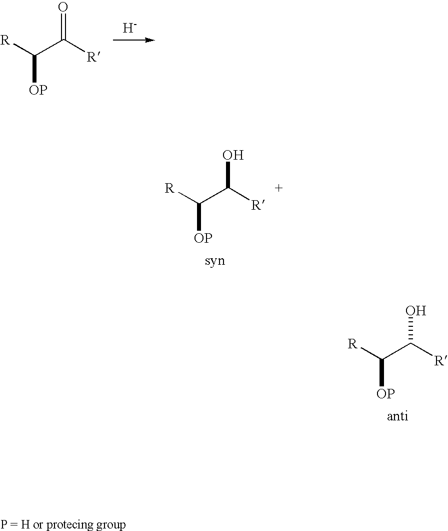

We will consider each reduction in turn. While some of the reagents may be new to you, you should already be aware of the underlying concepts and models; if you are not then REVISE this area of Chemistry—it will be cropping up time and time again in this lecture course.

For Example See:

1. F. A. Carey, R. J. Sundberg, Advanced Organic Chemistry: Volume B, Plenum Press, New York, 1990 (3rd Edition), pp 241-244.
2. M. B. Smith, Organic Synthesis, McGraw-Hill, New York, 1994, pp 400-417.
3. E. L. Eliel, S. H. Wilen, Stereochemistry of Organic Compounds, Wiley, New York, 1994, pp 858-938 for an indepth discussion of this area of Chemistry
   II.B.1 Diastereoselective Formation of Anti-1,3-Diols

A number of methods have been developed for forming the anti-1,3-diol from the corresponding r-hydroxy-ketone. All rely on the so-called DIRECTED REDUCTION which takes advantage of the intramolecular hydride transfer through a well-defined 6-membered chair-like transition state (c.f. Meerwein-Ponndorf-Verley reduction).

II.B.1.i Davis' Intramolecular Hydrosilylation

S. Anwar, A. P. Davis, Tetrahedrom, 1984, 40, 2233-2238. 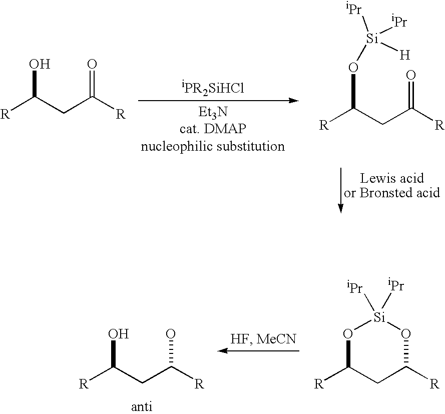
Step 1: form silyl ether
Step 2: Treat silane with Lewis or Brnsted acid to induce hydride transfer. Levels of diastereoselectivity are good to excellent anti:syn 320:1 to 120:1 (BF₃OEt₂ and SnCl₄ give particularly good results).
The silyl acetal product is stable and the isopropyl groups make this functionality a suitable diol protecting group.
Fluoride-induced deprotection of the silyl acetal provides the free diol.

What is the mechanism of fluoride induceds deprotection of silyl ethers? Hint. Silicon has low lying empty orbitals (3d AOs).

Intramolecular hydride transfer through a chair-like T.S. accounts for the stereochemical outcome of the reaction. 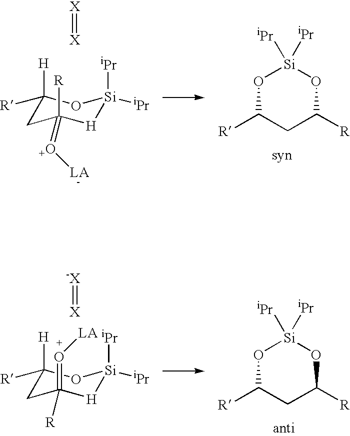
II.B.1.ii Tetramethylammonium Triacetoxyborohydride (Evans)

Evans has introduced an alternative approach using Me₄NHB(OAc)₃.

D. A. Evans, K. T. Chapman, E. M. Carreira, J Am. Chem. Soc., 1988, 110, 3560-3578.

Although the levels of selectivity are not as high as the Davis method the reaction is easier to perform and generally higher yielding (pay-off):

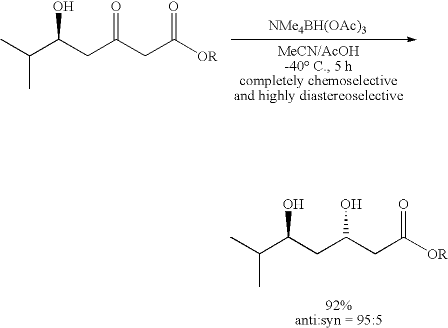

Note that only the r-ketone is reduced—the ester remains intact (chemoselective)

Draw a T.S. which satisfies the stereochemical outcome of the reaction (hint: the AcOH co-solvent provides acid catalysis)

II.B.1.iii Evans-Tishchenko Reduction

D. A. Evans, A. H. Hoveyda, J. Am. Chem. Soc., 1990, 112, 6447-6449.
provides anti-1,3-diol with high levels of stereocontrol
one potential advantage is that the directing hydroxyl group is protected as an ester (the choice of aldehye determines the nature of the PG)
this allows differentiation of two secondary alcohols which is sometimes difficult to achieve starting from the 1,3-diol.

The mechanism involves the reaction of a r-hydroxy ketone with an aldehyde (source of acyl protecting group) and is mediated by samarium diiodide (SmI₂). The samarium ensures the formation of a well-defined transition state (by coordination—recall that lanthanides are strongly oxophilic) and directs the transfer of hydride from the aldehyde to the ketone.

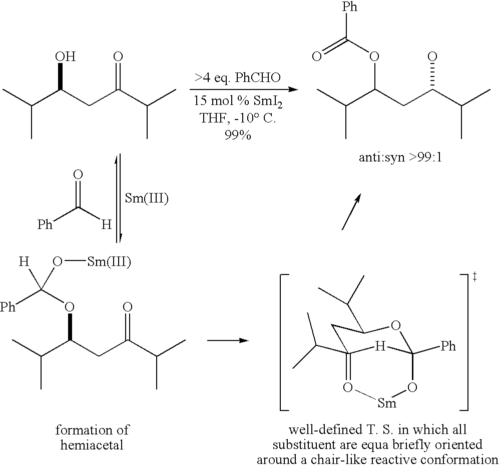

Q? How could you prove that the source of hydride is the aldehyde?
Another Example: 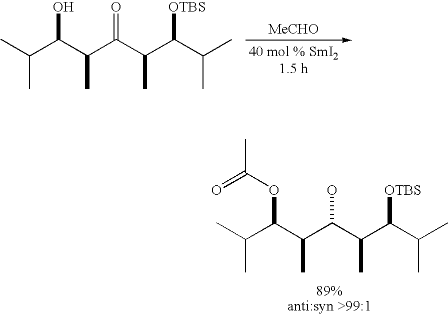
II.B.2 Diastereoselective Formation of Syn-1,3-Diols
Chelate-Controlled Intermolecular Hydride Delivery

Metals capable of forming a chelate between the r-hydroxyl group and ketone provide a molecular conformation which resembles that of cyclohexene: 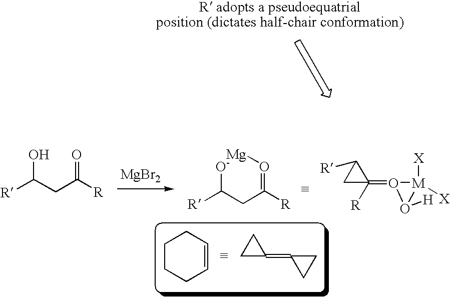
INTERmolecular hydride delivery on the chelate would then be expected to provide the syn-1,3-diol products. This is indeed the case.
The most reliable reaction conditions are Et₂B(OMe)—NaBH₄ at low temperature:

K.-M. Chen, G. E. Hardtmann, K. Prasad, O. Repic, M. J. Shapiro, Tetrahedron Lett., 1987, 28, 155-158.

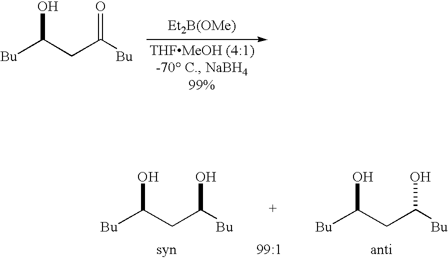

Make sure that you can rationalise the stereochemical outcome of this reaction using clear conformational diagrams.
other reagents which also give good syn selectivity are Zn(BH₄)₂ and DIBALH

K. Narasaka, F.-C. Pai, Tetrahedron, 1984, 40, 2233-2238.

There are numerous variants on this theme (internal chelation followed by intermolecular hydride delivery). For an example in which an ester is used to form the chelate:

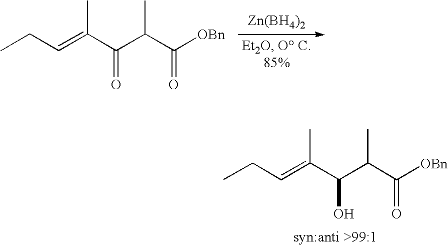

Draw a T.S. diagram which accounts for the observed stereochemical outcome of this reaction.

II.B.3 Diastereoselective Formation of Anti-1,2-Diols

Exploit Chelation Control:

therefore require:
   a free alcohol or a protected alcohol in which the protecting group can still form a chelate (alkyl ethers).
   a metal which can form a chelated intermediate (typical metals include Zn(II), Mg(II), Ti(IV) etc.)

Once again the chelated intermediate is much more conformationally rigid and sterically differentiates the two diastereotopic faces of the carbonyl group. [This is Cram chelation] 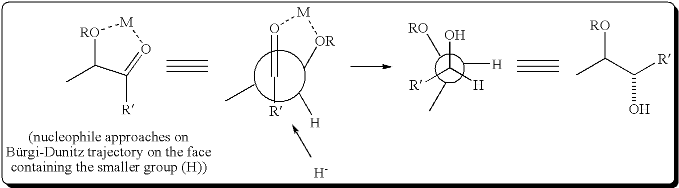
Examples: 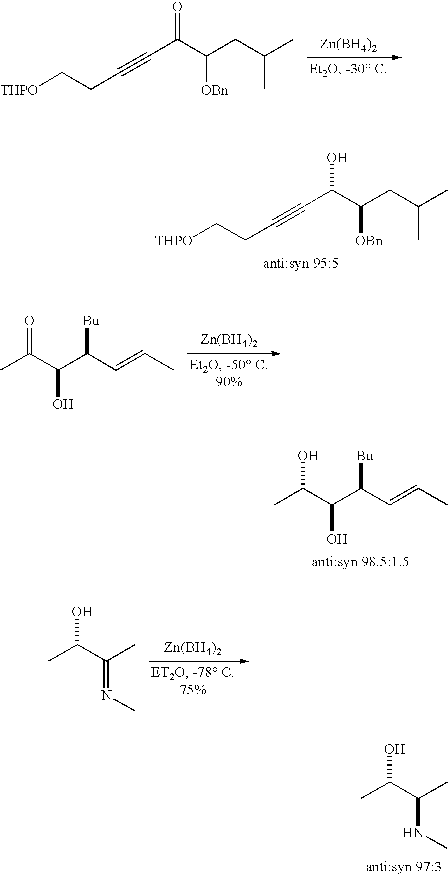
II.B.4 Diastereoselective Formation of Syn-1,2-Diols
This Requires:
careful choice of protecting group; one which supresses chelate formation and is very bulky (large silyl protecting groups are ideal).
use Felkin-Anh T.S. analysis to account for the stereocontrol.

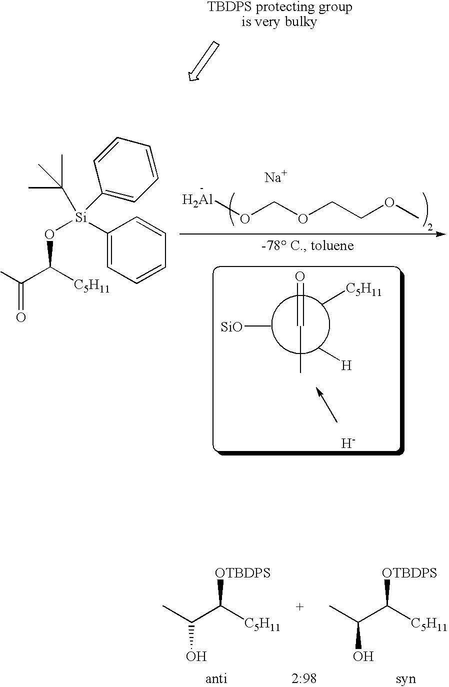

Make sure you understand the steric AND stereoelectronic arguments behind the Felkin-Anh T.S.

For Other Examples:

1. T. Takahashi, M. Miyazawa, J. Tsuji, Tetrahedron Lett., 1985, 26, 5139-5142.
2. L. E. Overman, R. J. McCready, Tetrahedron Lett., 1982, 23, 2355-2358.
   II.C Other Methods of Reduction
   II.C.1 Raney-Nickel
   most widely used in the hydrogenolysis of C—S bonds.
      Examples: 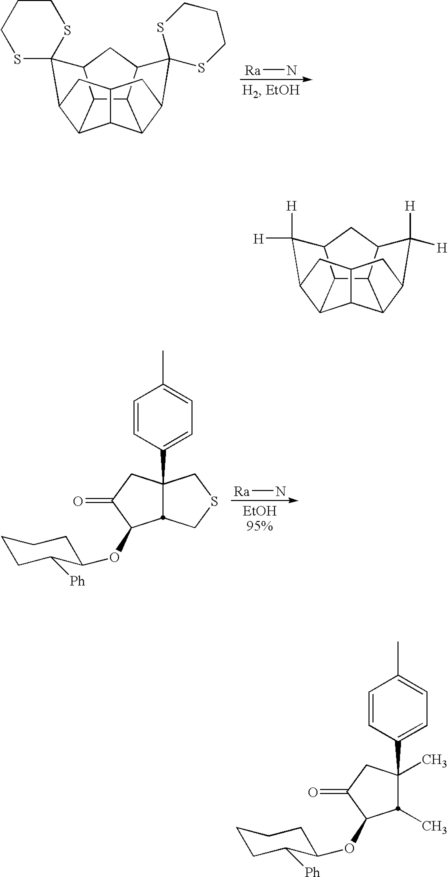
   also used in the hydrogenation of alkenes and alkynes.
      II.C.2 Zinc in Acidic Media
      Reduction of a-Haloketones
   very mild
   highly chemoselective
      Example:

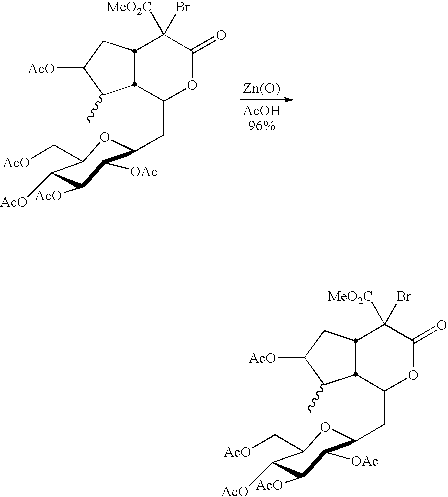

Note the lactone, acetate, glycosidic linkage and acetal all remain intact.

Q? What is the mechanism of reduction? Hint: the reaction involves single electron transfer.

1,4-Reduction of Enones

Example:

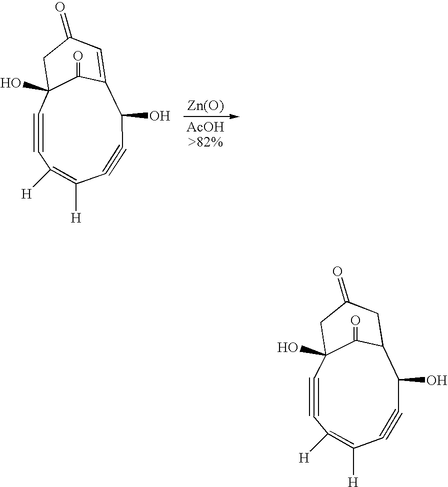

Note that there is a zinc enolate intermediate; this reaction can therefore be used for regioselective formation of enolates.

Clemmenson Reduction

A classical method for complete reduction of a carbonyl group (in ketones and aldehydes).
Reaction conditions are fairly vigorous.
   Example: 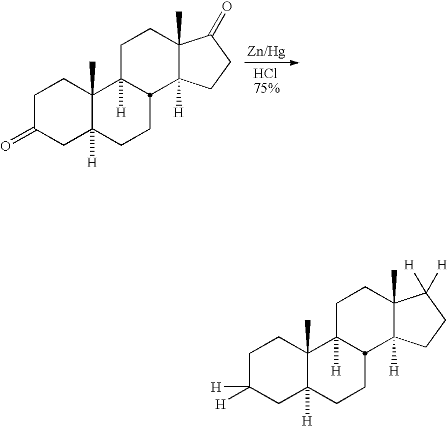
   II.D Hydrogenation with Hydrogen and a Transition Metal Catalyst
Typical catalysts are Pt, Pd, Rh, Ru and Ni (late transition metals)—usually used as finely dispersed solids or adsorbed on to an inert support such as charcoal or alumina.
Reaction takes place on the surface of the metal—heterogeneous catalysis.
Hydrogen is invariably transferred on to the less hindered face in a syn addition process.
   Example: 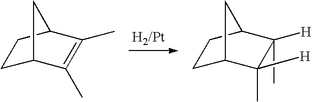
A variety of homogeneous catalysts are also effective e.g. Wilkinson's catalyst [(PPh₃)₃RhCl]
Transition metal-catalysts in the presence of H₂ will reduce carbonyl groups although the rate is usually lower than the reduction of olefins (allows chemoselectivity).
   Example:

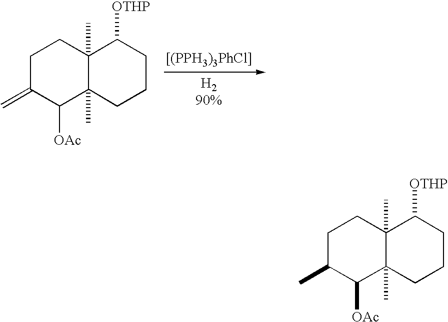

Q? How does the shape of the bicycle control the stereoselectivity of the hydrogenation?

Enantioselective reduction will NOT be discussed here. II.D.1 Partial Reduction of Alkynes
a useful route to (Z)-alkenes
need to modify the catalyst to minimise over-reduction
Lindlar's catalyst (Pd—CaCO₃—PbO) is the most widely used. The PbO tempers the reactivity of the catalyst by acting as a catalyst poison.
Other systems include Pd—BaSO₄ poisoned with quinoline.
   Example: 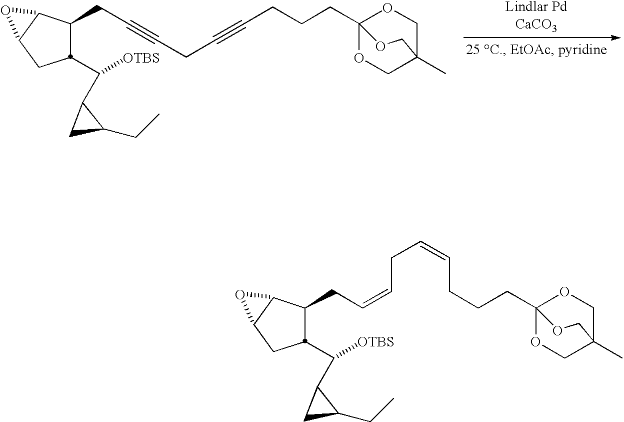
   II.D.2 Hydrogenolysis
Benzyl ethers are readily cleaved by Pd/C/H₂ to provide the free alcohol and toluene.
Cleavage occurs under mild and neutral conditions.
As a result, benzyl ethers are frequently used as alcohol protecting groups. 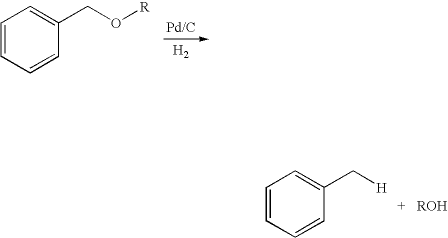
   II.E Dissolving Metal Reductions (Sodium/Ammonia or Lithium/Ammonia)
A wide variety of uses, only three will be discussed here.
Reactions proceed via single electron transfer processes.
   II.E.i Regiospecific Enolate Formation

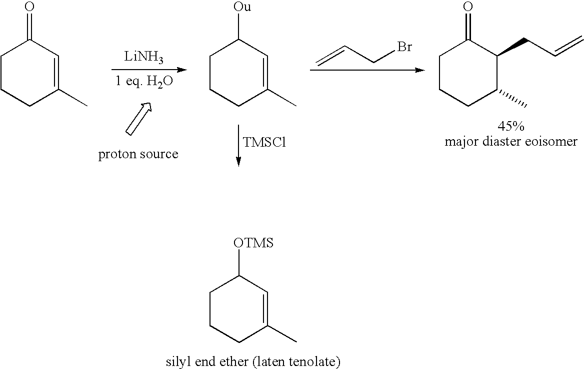

Enolates are ambident nucleophiles—you should be able to account for the differing regioselectivity of the reactions of the intermediate llithium enolate with the two different electrophiles.

II.E.2 Birch Reduction

Partial Reduction of Aromatic Rings

Mechanism: 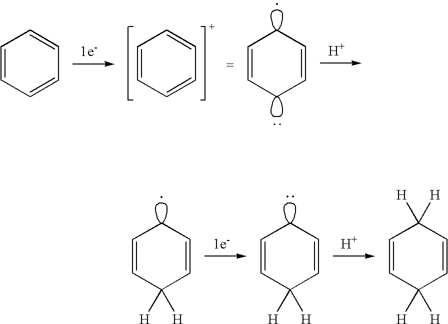
Under the (relatively controlled and mild) reaction conditions, reduction stops at the dihydro stage.
The rate of reduction is influenced by the substituents on the ring—as the intermediates are negatively charged, the rate is, not surprisingly, increased by electron withdrawing substituents.
Substituents also dictate the regiochemistry of protonation:

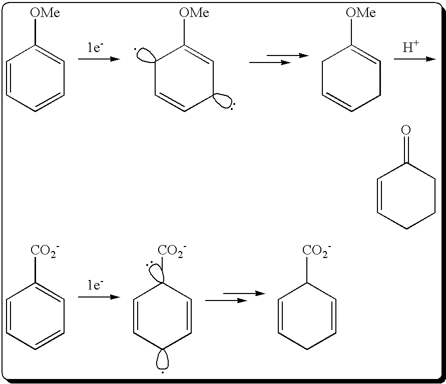

Make sure you can rationalise the regiochemistry of these reactions.

Reduction of Alkynes

a useful route to (E)-alkenes
equilibration of the radical or radical anionic intermediates ensures the thermodynamically more stable alkene is produced (usually the (E)-alkene).
   Mechanism: 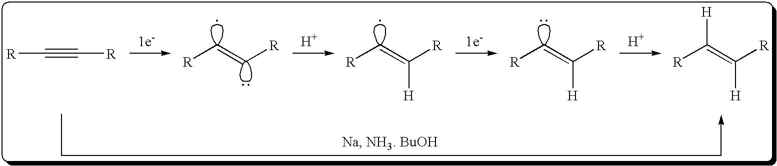
   II.F Free Radical Reductions
used to reduce alkyl halides
usual hydrogen atom donor is tributyltin hydride (Bu₃SnH)
   Mechanism: 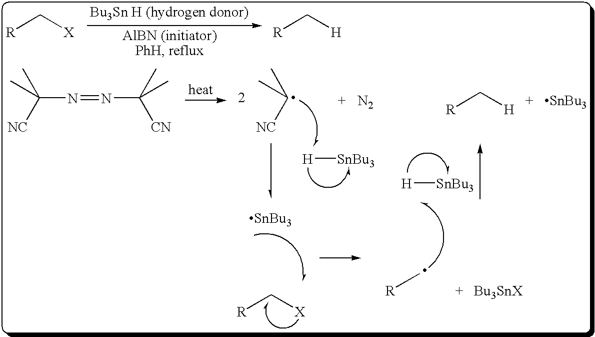
   Some Examples: 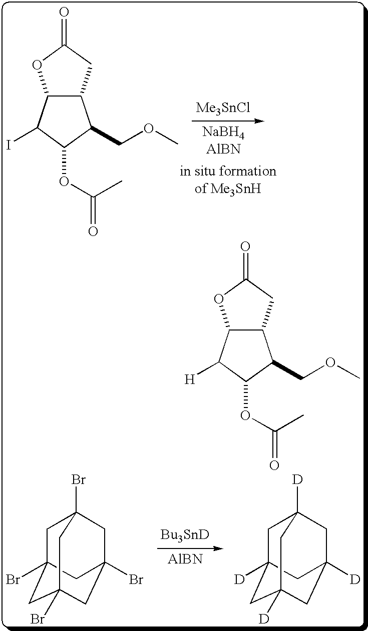
   Deoxygenation of Thioesters:

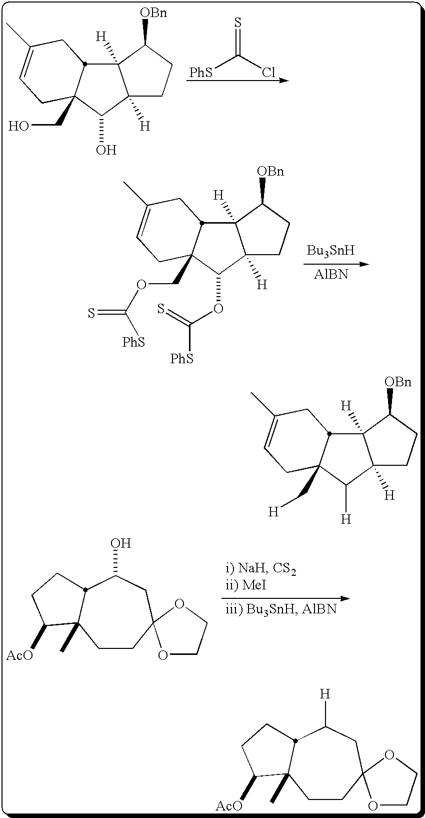

Q? What is the mechanism of this reaction? Hint: the driving force is formation of a C═O bond

### SUMMARY

In this section we have discussed a variety of methods for reducing carbonyl groups chemo-, regio- and stereoselectively and seen that this has necessitated the development of a wide variety of reducing agents. Understanding the mechanisms of various reducing agents allows a good method for predicting their reactivity towards potentially reactive functionality. We have also discussed various methods for reducing unsaturated compounds (olefins, alkynes and aromatic compounds) and seen the importance of late transition metals as catalysts in such reactions. Reduction requires the gain of electrons; metals are a potential source of electrons. We have seen that Zinc in acidic media and Li or Na in NH3 are good reducing systems. Free radical reduction occupies a special niche; it is particularly useful for reducing halides and similar systems under mild, neutral conditions.

1-28. (canceled)

29. A method of modifying an antigen to modify the Th2-type bias of the Th1/Th2-type immune response of an animal exposed to the antigen, the method comprising selectively:
(i) decreasing the number of reactive carbonyl groups present in the antigen so as to decrease the Th2-type bias; or,
(ii) increasing the number of reactive carbonyl groups present in the antigen so as to increase the Th2-type bias.

30. A method as in claim 29 wherein the animal is a human.

31. A method as in claim 29 wherein the antigen is or comprises an entity selected from the group consisting of a protein, glycoprotein, lipoprotein, polysaccharide, or a nucleic acid.

32. A method as in claim 29 wherein the antigen or a part thereof is derived from an entity selected from the group consisting of a mammalian cell, a plant cell, bacteria, virus, fungus or parasite.

33. A method as in claim 29 wherein the antigen or a part thereof is derived from a tumor or an autoantigen.

34. A method as in claim 29 wherein the antigen is a vaccine or vaccine component.

35. A method as in claim 34 wherein the vaccine or vaccine component has been formaldehyde-treated prior to being modified and wherein the vaccine or vaccine component is modified according to (i) in which the number of reactive carbonyl groups is decreased.

36. A method as in claim 29 wherein the antigen is modified by decreasing the number of reactive carbonyl groups and wherein the antigen is present in a foodstuff.

37. A method as in claim 36 wherein said foodstuff in which the number of reactive carbonyl groups is reduced is adapted to be incorporated into a product selected from processed foods, preserved foods, baby food, ready meals or to be applied to the skin.

38. A method as in claim 36 wherein said foodstuff is roasted nuts.

39. A method as in claim 29 wherein the decrease in the number of reactive carbonyl groups present in the antigen is effected by reduction with reducing agents.

40. A method as in claim 29 wherein the decrease in the number of reactive carbonyl groups present in the antigen is effected by hydrogenation.

41. A method as in claim 29 wherein the increase in the number of reactive carbonyl groups present in the antigen is effected by a treatment selected from the group consisting of aldehyde or formaldehyde treatment, oxidation and Maillard reaction.

42. A vaccine or vaccine component modified by the method of claim 1.

43. A vaccine or vaccine component as in claim 42 wherein the vaccine or vaccine component has been formaldehyde-treated prior to a reduction in the number of reactive carbonyl groups present.

44. A method as in claim 34 wherein the vaccine or vaccine component is for an organism selected from the group consisting of Respiratory Syncytial Virus, Measles, Influenza, human metapneumavirus, Hantaviruse (causative agent of haemorrhagic fever with renal syndrome (HFRS)), WEE, EEE, VEE (Western, Eastern and Venezuelan Equine Encephalitis), encephalitis viruses, anthrax, mumps, pertussis, viral hepatitis, meningitis, poliomyelitis, tuberculosis, rubella, tetanus, diphtheria or coronavirus infections.

45. A method as in claim 35 wherein the vaccine or vaccine component is for an organism selected from the group consisting of Respiratory Syncytial Virus, Measles, Influenza, human metapneumavirus, Hantaviruse (causative agent of haemorrhagic fever with renal syndrome (HFRS)), WEE, EEE, VEE (Western, Eastern and Venezuelan Equine Encephalitis), encephalitis viruses, anthrax, mumps, pertussis, viral hepatitis, meningitis, poliomyelitis, tuberculosis, rubella, tetanus, diphtheria or coronavirus infections.

46. A vaccine or vaccine component as in claim 42 wherein the vaccine or vaccine component is for an organism selected from the group consisting of Respiratory Syncytial Virus, Measles, Influenza, human metapneumavirus, Hantaviruse (causative agent of haemorrhagic fever with renal syndrome (HFRS)), WEE, EEE, VEE (Western, Eastern and Venezuelan Equine Encephalitis), encephalitis viruses, anthrax, mumps, pertussis, viral hepatitis, meningitis, poliomyelitis, tuberculosis, rubella, tetanus, diphtheria or coronavirus infections.

47. A vaccine or vaccine component as in claim 43 wherein the vaccine or vaccine component is for an organism selected from the group consisting of Respiratory Syncytial Virus, Measles, Influenza, human metapneumavirus, Hantaviruse (causative agent of haemorrhagic fever with renal syndrome (HFRS)), WEE, EEE, VEE (Western, Eastern and Venezuelan Equine Encephalitis), encephalitis viruses, anthrax, mumps, pertussis, viral hepatitis, meningitis, poliomyelitis, tuberculosis, rubella, tetanus, diphtheria or coronavirus infections.

48. A vaccine or vaccine component as in claim 42 wherein the vaccine or vaccine component has been modified to increase the number of reactive carbonyl groups present.

49. A foodstuff modified by the method of claim 1.

50. A foodstuff as in claim 49 wherein said foodstuff is roasted nuts.

51. A composition selected from the group consisting of an antigen modified by the method of claim 1 and a vaccine or vaccine component according to claim 42 and, optionally, an adjuvant.

52. A composition as in claim 51 and a pharmaceutically acceptable carrier as contained in a pharmaceutical composition.

53. A method as in claim 29 comprising exposing an animal to said modified antigen to decrease the Th2-type bias of the Th1/Th2-type immune response of said animal.

54. A method as in claim 29 wherein a treatment selected from the group consisting of aldehyde treatment, formaldehyde treatment, oxidation or Maillard reaction is used to modify an antigen to increase the Th2-type bias of the Th1/Th2-type immune response of an animal exposed to the antigen.

55. A kit of parts comprising an antigen and an agent selected from the group consisting of a reducing agent capable of decreasing the number of reactive carbonyl groups on the antigen or aldehyde, formaldehyde, oxidation or an agent for catalyzing a Maillard reaction to increase the number of reactive carbonyl groups on the antigen and, optionally, an adjuvant and/or a pharmaceutically acceptable carrier.
